(19) United States
(12) Patent Application Publication
(10) Pub. No.: US 2026/0347926 A1
(43) Pub. Date: Nov. 5, 2026

(54) METHODS FOR MITIGATION OF METHYLATION BIAS

(71) Applicant: Foundation Medicine, Inc., Boston, MA (US)

(72) Inventors: Nicole LAMBERT, San Diego, CA (US); James YU, San Diego, CA (US); Neil PETERMAN, San Francisco, CA (US); Yexun WANG, San Diego, CA (US); Alexander ROBERTSON, Princeton, NJ (US); Jason CLOSE, Cambridge, MA (US)

(73) Assignee: Foundation Medicine, Inc., Boston, MA (US)

(21) Appl. No.: 19/486,860

(22) PCT Filed: May 23, 2024
(86) PCT No.: PCT/US2024/030762

§ 371 Date: Nov. 21, 2025

Related U.S. Application Data
(60) Provisional application No. 63/468,742, filed on May 24, 2023.

(51) Int. Cl. C12Q 1/6869; C12Q 1/25; C12Q 1/48; C12Q 1/6806; C12Q 1/6813; C12Q 1/6855; C12Q 1/6886; C40B 40/06; C40B 50/06; C40B 70/00; G16B 30/00

(52) U.S. Cl. CPC C12Q 1/6869; C12Q 1/25; C12Q 1/48; C12Q 1/6806; C12Q 1/6813; C12Q 1/6855; C12Q 1/6886; C12Y 207/07007; C12Y 605/01001; C12Y 605/01002; C40B 40/06; C40B 50/06; C40B 70/00; G16B 30/00; C12Q 2600/106; C12Q 2600/154; G01N 2800/52; G01N 2800/7028

(57) ABSTRACT

Methods for mitigation of methylation bias during preparation of sequencing libraries are described. The methods may comprise, e.g., extracting DNA fragments from a sample; performing at least one of: (i) a first end repair reaction, where the first end repair reaction comprises the use of a non-strand-displacing DNA polymerase; or (ii) a second end repair reaction, where the second end repair reaction comprises the use of a chain termination mechanism; performing at least one of: (iii) a tailing reaction to add an overhanging polynucleotide strand to the end-repaired DNA fragments, where the tailing reaction comprises the use of a single deoxynucleotide triphosphate (dNTP); or (iv) a nick/gap repair reaction using a DNA ligase to fill in single-stranded nicks or gaps in the end-repaired DNA fragments, to generate a plurality of modified DNA fragments; and performing methylation analysis of nucleic acid molecules derived from the plurality of modified DNA fragments.

### CROSS-REFERENCE TO RELATED APPLICATION

This application claims the priority benefit of U.S. Provisional Patent Application Ser. No. 63/468,742, filed May 24, 2023, the contents of which are incorporated herein by reference in their entirety.

### FIELD OF THE INVENTION

The present disclosure relates generally to methods for analyzing genomic profiling data, and more specifically to methods for mitigating methylation bias when preparing DNA sequencing libraries for performing nucleic acid sequencing and methylation analysis.

### BACKGROUND

In preparation for sequencing, nucleic acid molecules extracted from a sample (e.g., a patient sample or a sample from a subject) are subjected to certain library preparation methods. In the case of Next Generation Sequencing, for example, the library preparation methods may involve ligating instrument-specific DNA sequences (adapters) to each end of each of fragments of the nucleic acid molecules obtained from the sample. However, the extracted nucleic acid molecules (e.g., DNA), or fragments thereof, that are typically used as input for sequencing library preparation often have overhangs containing single stranded DNA (ssDNA), breaks in the phosphodiester backbone that exist on just one strand (nicks), and/or ssDNA regions internal to the duplex molecule (gaps). All of these features must be fixed during library preparation (e.g., end repair and end modification) in order to capture and sequence the DNA extracted from the sample in its entirety. Typical sequencing library preparation protocols comprise fixing nicks, gaps, and overhangs using a combination of 3′ exonuclease digestion to remove 3′ overhangs and nick/gap filling using a strand displacing polymerase, which results in a blunt-ended, double stranded DNA (dsDNA) molecule. Although the identities of the DNA bases within a given fragment are mostly preserved in this process, epigenetic markers such as cytosine methylation are often lost. The artificial loss of cytosine methylation during library preparation is known a “methylation bias” (or “M-bias”), and has a negative impact on the ability to accurately assess methylation status based on sequence read data. Methylation bias thus also has a negative impact on the ability to use methylation status (e.g., a methylation signature for a sample from a subject) as a biomarker for diagnosis of disease and/or prognosis of healthcare outcomes. Thus, improved methods for sequencing library preparation are required, particularly when the resulting library is to be sequenced as part of evaluating the methylation status of the subject's DNA.

### BRIEF SUMMARY OF THE INVENTION

Disclosed herein are methods for mitigating methylation bias during preparation of DNA libraries for use in performing nucleic acid sequencing and for performing sequence read-based methylation analysis of a sample collected from a subject (e.g., a patient). The disclosed methods comprise the use of library preparation steps that prevent strand displacement by polymerases and/or block resynthesis activity by polymerases during end repair and modification to prevent incorporation of unmethylated cytosines in place of methylated cytosines. The disclosed methods enable more accurate determinations of methylation status based on sequence read data, and may improve the ability to detect hypomethylation biomarkers associated with disease, e.g., cancer.

Disclosed herein are methods comprising: extracting one or more nucleic acid fragments from a sample obtained from a subject; performing at least one of: a first end repair reaction to repair fragmentation damage to one or more nucleic acid fragment ends, wherein the first end repair reaction comprises the use of a non-strand-displacing polymerase; or a second end repair reaction to repair fragmentation damage to one or more nucleic acid fragment ends, wherein the second end repair reaction comprises the use of a chain termination mechanism; performing at least one of: a tailing reaction to add an overhanging polynucleotide strand to the one or more end-repaired nucleic acid fragments, wherein the tailing reaction comprises the use of a single deoxynucleotide triphosphate (dNTP); or a nick/gap repair reaction to fill in single-stranded nicks or gaps in the one or more end-repaired nucleic acid fragments, wherein the nick/gap repair reaction comprises the use of a ligase to generate a plurality of modified nucleic acid fragments; and performing methylation analysis of the plurality of modified nucleic acid fragments.

In some embodiments, the methylation analysis comprises performing a cytosine conversion reaction on the plurality of modified nucleic acid fragments to generate a plurality of converted nucleic acid fragments. In some embodiments, the methylation analysis comprises sequencing the plurality of converted nucleic acid fragments to generate a plurality of sequence reads. In some embodiments, the method further comprises performing a sequence read analysis based on the plurality of sequence reads to determine a methylation signature of the subject.

In some embodiments, the method comprises performing both a first end repair reaction and a second end repair reaction. In some embodiments, the method comprises performing both a tailing reaction comprising the use of a single dNTP and a nick/gap repair reaction comprising the use of a ligase.

In some embodiments, the methylation analysis comprises a restriction enzyme-based, affinity enrichment-based, bisulfite conversion-based, or enzymatic conversion-based methylation sequencing method.

In some embodiments, the method further comprises ligating one or more adapters to the plurality of modified nucleic acid fragments. In some embodiments, the one or more adapters comprise one or more adapters comprising an overhanging poly-T sequence. In some embodiments, the one or more adapters comprise one or more sequencing adapters. In some embodiments, the one or more sequencing adapters comprise one or more methylated stubby adapters, flow cell adapters, read 1 sequencing adapters, read 2 sequencing adapters, or any combination thereof. In some embodiments, the one or more adapters comprise one or more barcodes.

In some embodiments, the method further comprises ligating one or more barcodes to the plurality of modified nucleic acid fragments. In some embodiments, the one or more barcodes comprise a library index, a sample barcode, a cell barcode, a target-specific barcode, a DNA fragment barcode, a unique molecular index, or any combination thereof.

In some embodiments, the cytosine conversion reaction is used to convert non-methylated cytosine to uracil. In some embodiments, the cytosine conversion reaction comprises a chemical conversion reaction. In some embodiments, the chemical conversion reaction comprises a bisulfite conversion reaction. In some embodiments, the cytosine conversion reaction comprises an enzymatic conversion reaction. In some embodiments, the enzymatic conversion reaction comprises the use of a tet methylcytosine dioxygenase 2 (TET2) enzyme to oxidize 5-methyl-cytosine (5mC) or 5-hydroxymethyl-cytosine (5hmC) to 5-carboxycytosine (5caC). In some embodiments, the enzymatic conversion reaction further comprises the use of a combination of TET2 and T4 β-glucosyltransferase (T4-βGT) enzymes to convert 5-methyl-cytosine (5mC) or 5-hydroxymethyl-cytosine (5hmC) to 5-(D-glucosyl)oxymethyl-cytosine. In some embodiments, the enzymatic conversion reaction comprises the use of an Apolipoprotein B mRNA Editing Catalytic Polypeptide-like (APOBEC) enzyme.

In some embodiments, the method further comprises performing a nucleic acid amplification reaction to generate amplified nucleic acid molecules that are derived from the plurality of modified nucleic acid fragments. In some embodiments, the nucleic acid amplification reaction comprises a polymerase chain reaction (PCR). In some embodiments, the nucleic acid amplification reaction comprises a rolling circle amplification (RCA) reaction. In some embodiments, the method further comprises capturing a subset of nucleic acid molecules from the amplified nucleic acid molecules and performing methylation analysis on the subset.

In some embodiments, the one or more nucleic acid fragments comprises double-stranded nucleic acid fragments.

In some embodiments, the non-strand-displacing polymerase comprises a non-strand-displacing DNA polymerase. In some embodiments, the non-strand displacing DNA polymerase comprises T4 DNA polymerase.

In some embodiments, the chain termination mechanism comprises the use of a chain termination nucleotide in the end repair reaction. In some embodiments, the chain termination nucleotide comprises a 2′,3′-dideoxyribonucleoside 5′-triphosphate (ddNTP). In some embodiments, the ddNTP comprises a 2′,3′-dideoxycytidine 5′-triphosphate (ddCTP), 2′,3′-dideoxyguanosine 5′-triphosphate (ddGTP), 2′,3′-dideoxythymidine 5′-triphosphate (ddTTP), 2′,3′-dideoxyadenosine 5′-triphosphate (ddATP), or any combination thereof. In some embodiments, a ratio of ddNTP concentration to a corresponding dNTP concentration used to perform the end repair reaction is less than 20×, 30×, 40×, 50×, or 60×, 70×, 80×, 100×, 120×, or 140×.

In some embodiments, the chain termination mechanism comprises omitting a dNTP from the end repair reaction. In some embodiments, the chain termination mechanism comprises including only a limiting amount of a dNTP in the end repair reaction.

In some embodiments, one or more modified nucleotides are used as part of performing the end repair reaction to indicate nucleic acid sequence regions where end repair has occurred. In some embodiments, one or more modified nucleotides are used as part of performing the tailing reaction to identify the overhanging poly-nucleotide strand added to the modified nucleic acid fragments. In some embodiments, one or more modified nucleotides are used as part of performing the nick/gap repair reaction to identify filled in portions of the modified nucleic acid fragments. In some embodiments, the one or more modified nucleotides comprise 5-methyldeoxycytidine 5′-triphosphate (5-methyl dCTP), 5-hydroxymethyldeoxycytidine 5′-triphosphate, deoxyuradine 5′-triphosphosphate, oxoguanosine 5′-triphosphate, or any combination thereof.

In some embodiments, the tailing reaction comprises the use of 2′-deoxyadenosine 5′-triphosphate (dATP). In some embodiments, the tailing reaction comprises the use of 2′-deoxythymidine 5′-triphosphate (dTTP). In some embodiments, the tailing reaction comprises the use of 2′-deoxycytidine 5′-triphosphate (dCTP). In some embodiments, the tailing reaction comprises the use of 2′-deoxyguanosine 5′-triphosphate (dGTP). In some embodiments, tailing reaction comprises the use of T4 DNA polymerase, T4 Polynucleotide Kinase Reaction (T4 PNK), Taq polymerase, Klenow Fragment (3′→5′ exo-), Sulfolobus DNA polymerase IV, or any combination thereof. In some embodiments, the tailing reaction is omitted and a downstream blunt ligation step is used to ligate one or more adapters to the one or more nucleic acid fragments.

In some embodiments, the ligase comprises a DNA ligase. In some embodiments, the DNA ligase comprises Taq DNA ligase, T4 DNA ligase, 9oN□ DNA ligase, T3 DNA ligase, or any combination thereof.

In some embodiments, sequence read data obtained by sequencing nucleic acid molecules derived from the plurality of modified nucleic acid fragments exhibits reduced methylation bias compared to that obtained by sequencing a conventionally-prepared DNA sequencing library. In some embodiments, the reduction in methylation bias is greater than 5%, 10%, 15%, 20%, 25%, 30%, or 35% as measured by standard deviation (SD) methyl position bias.

In some embodiments, the methylation analysis is performed using a next-generation sequencer. In some embodiments, the methylation analysis further comprises sequencing, using the next-generation sequencer, the plurality of modified nucleic acid fragments. In some embodiments, the methylation analysis comprises using the next-generation sequencing to perform whole genome sequencing, whole exome sequencing, or targeted sequencing. In some embodiments, the method further comprises determining a methylation status for each of one or more genomic loci based on sequence read data for the plurality of sequence reads.

In some embodiments, the method further comprises screening, detecting, diagnosing, confirming a diagnosis of, or monitoring disease in the subject based on sequence read data for the plurality of sequence reads. In some embodiments, the screening, detecting, diagnosing, confirming a diagnosis, or monitoring of disease is performed with improved accuracy due to a reduction in methylation bias in the sequence read data compared to that obtained by sequencing a conventionally-prepared DNA sequencing library. In some embodiments, the method further comprises detecting minimum residual disease in the subject based on sequence read data for the plurality of sequence reads. In some embodiments, the disease is cancer. In some embodiments, the methylation analysis is used to detect hypomethylated or hypermethylated genomic regions in cancer patients.

In some embodiments, the one or more nucleic acid fragments comprise one or more DNA fragments.

Also disclosed herein are methods comprising: extracting a plurality of DNA fragments from a sample obtained from a subject; performing at least one of: a first end repair reaction to repair fragmentation damage to one or more DNA fragment ends, wherein the first end repair reaction comprises the use of a non-strand-displacing DNA polymerase; or a second end repair reaction to repair fragmentation damage to one or more DNA fragment ends, wherein the second end repair reaction comprises the use of a chain termination mechanism; performing at least one of: a tailing reaction to add an overhanging polynucleotide strand to the one or more end-repaired DNA fragments, wherein the tailing reaction comprises the use of a single dNTP; or a nick/gap repair reaction to fill in single-stranded nicks or gaps in the one or more end-repaired DNA fragments, wherein the nick/gap repair reaction comprises the use of a DNA ligase to generate a plurality of modified DNA fragments; ligating one or more adapters onto one or more modified DNA fragments from the plurality of modified DNA fragments; performing a cytosine conversion reaction on the one or more ligated DNA fragments to generate one or more converted DNA fragments; amplifying the one or more converted DNA fragments; capturing one or more amplified converted DNA fragments; sequencing, by a sequencer, the one or more captured converted DNA fragments to obtain a plurality of sequence reads that represent the one or more captured converted DNA fragments; receiving, at one or more processors, sequence read data for the plurality of sequence reads; and performing a methylation analysis of nucleic acid molecules derived from the plurality of modified DNA fragments based on the plurality of sequence reads. In some embodiments, the methylation analysis comprises determining a methylation signature for the subject.

In some embodiments, the method comprises performing both a first end repair reaction and a second end repair reaction. In some embodiments, the method comprises performing both a tailing reaction comprising the use of a single dNTP and a nick/gap repair reaction comprising the use of a DNA ligase.

In some embodiments, the subject is suspected of having or is determined to have cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprises treating the subject with an anti-cancer therapy. In some embodiments, the anti-cancer therapy comprises a targeted anti-cancer therapy. In some embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of DNA fragments comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, sequencing adapter sequences, flow cell adaptor sequences, substrate adapter sequences, or sample index sequences.

In some embodiments, the captured DNA fragments are captured from the amplified DNA fragments by hybridization to one or more bait molecules. In some embodiments, the one or more bait molecules comprise one or more nucleic acid molecules, each comprising a region that is complementary to a region of a captured DNA fragment.

In some embodiments, amplifying converted DNA fragments comprises performing a polymerase chain reaction (PCR) amplification technique, a non-PCR amplification technique, or an isothermal amplification technique.

In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer.

In some embodiments, one or more of the plurality of sequencing reads overlap one or more gene loci within one or more subgenomic intervals in the sample. In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73,CDH1, CDK12, CDK4, CDK6,CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some embodiments, the method further comprises generating, by the one or more processors, a report indicating a result of the methylation analysis. In some embodiments, the method further comprising transmitting the report to a healthcare provider. In some embodiments, the report is transmitted via a computer network or a peer-to-peer connection.

Disclosed herein are methods for diagnosing a disease, the methods comprising: diagnosing that a subject has the disease based on a methylation analysis or a determination of a methylation signature of a sample from the subject, wherein the methylation analysis or the methylation signature is determined according to any of the methods described herein.

Disclosed herein are methods of selecting an anti-cancer therapy, the methods comprising: responsive to a methylation analysis or a determination of a methylation signature of a sample from a subject, selecting an anti-cancer therapy for the subject, wherein the methylation analysis or the methylation signature is determined according to any of the methods described herein.

Disclosed herein are methods of treating a cancer in a subject, comprising: responsive to a methylation analysis or a determination of a methylation signature of a sample from the subject, administering an effective amount of an anti-cancer therapy to the subject, wherein the methylation analysis or the methylation signature is determined according to any of the methods described herein.

Disclosed herein are methods for monitoring cancer progression or recurrence in a subject, the method comprising: performing a first methylation analysis or determining a first methylation signature for a first sample obtained from the subject at a first time point according to any of the methods described herein; performing a second methylation analysis or determining a second methylation signature for a second sample obtained from the subject at a second time point; and comparing the first methylation analysis or methylation signature to the second methylation analysis or methylation signature, thereby monitoring the cancer progression or recurrence. In some embodiments, the second methylation analysis or methylation signature for the second sample is determined according to any of the methods described herein. In some embodiments, the method further comprises selecting an anti-cancer therapy for the subject in response to the cancer progression. In some embodiments, the method further comprises administering an anti-cancer therapy to the subject in response to the cancer progression. In some embodiments, the method further comprises adjusting an anti-cancer therapy for the subject in response to the cancer progression. In some embodiments, the method further comprises adjusting a dosage of the anti-cancer therapy or selecting a different anti-cancer therapy in response to the cancer progression. In some embodiments, the method further comprises administering the adjusted anti-cancer therapy to the subject. In some embodiments, the first time point is before the subject has been administered an anti-cancer therapy, and wherein the second time point is after the subject has been administered the anti-cancer therapy.

In some embodiments, the subject has a cancer, is at risk of having a cancer, is being routine tested for cancer, or is suspected of having a cancer. In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments, the method further comprises determining, identifying, or applying a methylation analysis result or methylation signature for the sample as a diagnostic value associated with the sample.

In some embodiments, the method further comprises generating a genomic profile for the subject based on the determination of a methylation analysis result or methylation signature. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test. In some embodiments, the method further comprises selecting an anti-cancer therapy, administering an anti-cancer therapy, or applying an anti-cancer therapy to the subject based on the generated genomic profile.

In some embodiments of any of the methods described herein, the determination of a methylation analysis result or a methylation signature for the sample is used in making suggested treatment decisions for the subject. In some embodiments, the determination of a methylation analysis result or a methylation signature for the sample is used in applying or administering a treatment to the subject.

Also disclosed herein are methods comprising: extracting a plurality of nucleic acid molecules from a sample obtained from a subject; performing at least one of: a first end repair reaction to repair fragmentation damage to one or more ends of the nucleic acid molecules, wherein the first end repair reaction comprises the use of a non-strand-displacing DNA polymerase; or a second end repair reaction to repair fragmentation damage to the one or more ends, wherein the second end repair reaction comprises the use of a chain termination mechanism; performing at least one of: a tailing reaction to add an overhanging polynucleotide strand to the one or more end-repaired nucleic acid molecules, wherein the tailing reaction comprises the use of a single deoxynucleotide triphosphate (dNTP); or a nick/gap repair reaction to fill in single-stranded nicks or gaps in the one or more end-repaired nucleic acid molecules, wherein the nick/gap repair reaction comprises the use of a DNA ligase to generate a plurality of modified nucleic acid molecules; and performing sequence analysis of the modified nucleic acid molecules.

Disclosed herein are systems comprising: an extractor configured to extract a plurality of a plurality of nucleic acid molecules from a sample obtained from a subject; an automated library preparation device configured to execute one or more processes that when executed perform one or more of: a first end repair reaction to repair fragmentation damage to one or more ends of the nucleic acid molecules, wherein the first end repair reaction comprises the use of a non-strand-displacing DNA polymerase; or a second end repair reaction to repair fragmentation damage to the one or more ends, wherein the second end repair reaction comprises the use of a chain termination mechanism; performing at least one of: a tailing reaction to add an overhanging polynucleotide strand to the one or more end-repaired nucleic acid molecules, wherein the tailing reaction comprises the use of a single deoxynucleotide triphosphate (dNTP); or a nick/gap repair reaction to fill in single-stranded nicks or gaps in the one or more end-repaired nucleic acid molecules, wherein the nick/gap repair reaction comprises the use of a DNA ligase to generate a plurality of modified nucleic acid molecules; a sequencer configured to sequence the modified nucleic acid molecules and generate one or more sequence modified nucleic acid sequence reads representing the nucleic acid sequence of the modified nucleic acid molecule; and a computational analysis platform including one or more processors including a memory that stores one or more processes, the processes when executed configured to analyze the modified nucleic acid reads and identify one or more alterations or epigenetic signatures in the modified nucleic acid molecules.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A provides a non-limiting example of a process for preparing DNA sequencing libraries for methylation analysis, according to some embodiments disclosed herein.

FIG. 1B provides a non-limiting example of a process for preparing DNA sequencing libraries for methylation analysis, according to other embodiments disclosed herein.

FIG. 2 provides a non-limiting schematic illustration of end repair and nick/gap repair reactions performed during the preparation of DNA sequencing libraries.

FIG. 3 provides another non-limiting schematic illustration of end repair and nick/gap repair reactions performed during the preparation of DNA sequencing libraries, and the methylation artifacts that can arise as a result.

FIG. 4 depicts an exemplary computing device or system in accordance with one embodiment of the present disclosure.

FIG. 5 depicts an exemplary computer system or computer network, in accordance with some instances of the systems described herein.

FIG. 6 provides a non-limiting example of percent CpG methylation data that illustrates the effect of methylation bias in sequence read data.

FIG. 7 provides a non-limiting example of a box plot of data for observed methylation bias (quantified as standard deviation (SD) methylation position bias) with and without using Taq DNA ligase (in combination with a non-strand displacing polymerase) to perform nick or gap repair during library preparation.

FIGS. 8A-C provide non-limiting examples of data for hypomethylation signal (FIG. 8A), yield (FIG. 8B), and average methylation fraction (FIG. 8C) for eight different end repair and modification protocols that comprises the use of the Klenow fragment and mixtures of deoxycytidine triphosphate (dCTP) and dideoxycytidine triphosphate (ddCTP). FIG. 8D: schematic illustration of including ddCTP in the end repair and nick/gap repair reaction to mitigate methylation bias.

FIG. 9 provides a non-limiting example of data for SD methylation position bias observed using four different tailing reaction protocols comprising the use of either the KlenTaq DNA polymerase or Taq DNA polymerase in combination with either a dNTP mixture or dATP only.

FIG. 10A provides a non-limiting example of methylation bias data (as quantified by percent CpG methylation) observed in healthy/unaffected sample when sequencing libraries were prepared using the five different library preparation methods summarized in FIG. 10B.

FIG. 11 provides non-limiting examples of methylation bias data (quantified as SD methylation position bias) for sequence read data for DNA extracted from NSCLC and unaffected cfDNA samples using the five different library preparation methods summarized in the lower panel of the figure.

FIG. 12 provides non-limiting examples of hypomethylation score data calculated for the methylation bias data presented in FIG. 11.

FIG. 13 provides a non-limiting example of the ratio of hypomethylation score for NSCLC samples to that for healthy/unaffected samples (plotted on a log base 2 scale) for the five different library preparation methods summarized in the lower panel of the figure.

FIG. 14 provides a non-limiting example of percent CpG methylation as a function of sequence read position when DNA sequencing libraries were prepared using non-methylated dCTP or 5-methylated dCTP during end repair.

FIGS. 15A-B provide non-limiting examples of data for average methylation fraction (AMF) in healthy/unaffected sample for exemplary methylated control regions. FIG. 15A: plot of AMF versus sequence read position for Read 2. FIG. 15B: expanded scale plot of the data shown in FIG. 15A.

FIGS. 16A-B provide non-limiting examples of data for average methylation fraction (AMF) in healthy/unaffected sample for exemplary methylated control regions. FIG. 16A: plot of AMF versus sequence read position for Read 1. FIG. 16B: expanded scale plot of the data shown in FIG. 16A.

FIG. 17 provides non-limiting examples of methylation bias pattern data that illustrates that methylation bias is consistent in sequence read data derived from many different samples.

FIGS. 18A-D provide non-limiting examples of plots of baseline AMF background (FIG. 18A), slip rate (FIG. 18B), the number of end repair bases (FIG. 18C), and trimmed AUC (FIG. 18D) observed for sequence read data derived from samples for health individuals and individuals diagnosed with cancer.

FIGS. 19A-B provide non-limiting examples of data for nucleic acid yield for three different sample extracts plotted as a function of the conditions used to perform end repair and A tailing during sequencing library preparation. FIG. 19A: data for sample extract identification number OT04788. FIG. 19B: data for sample extract identification number OT04789. FIG. 19C: data for sample extract identification number OT04796.

FIG. 20 provides a non-limiting example of data for SD methylation position bias for three different sample extracts plotted as a function of the conditions used to perform end repair and A tailing during sequencing library preparation.

FIGS. 21A-C provide non-limiting examples of data for percent CpG methylation for three different sample extracts plotted as a function of sequence read position for the different conditions used to perform end repair and A tailing during sequencing library preparation. FIG. 21A: data for sample extract identification number OT04788. FIG. 21B: data for sample extract identification number OT04789. FIG. 21C: data for sample extract identification number OT04796.

FIGS. 22A-C provide non-limiting examples of data for slip rate for three different sample extracts plotted as a function of the conditions used to perform end repair and A tailing during sequencing library preparation. FIG. 22A: data for sample extract identification number OT04788. FIG. 22B: data for sample extract identification number OT04789. FIG. 22C: data for sample extract identification number OT04796.

FIG. 23 provides a non-limiting example of data for single base C-to-T substitution error rate plotted as a function of the conditions used to perform end repair and A tailing during sequencing library preparation.

FIGS. 24A-C provide a non-limiting example of data for erroneous error rates for C→T single base substitutions plotted for three different sample extracts as a function of the conditions used to perform end repair and A tailing during sequencing library preparation. FIG. 24A: data for sample extract identification number OT04788. FIG. 24B: data for sample extract identification number OT04789. FIG. 24C: data for sample extract identification number OT04796.

### DETAILED DESCRIPTION

Methods for mitigating methylation bias during preparation of DNA libraries for use in performing nucleic acid sequencing and for performing sequence read-based methylation analysis of a sample collected from a subject (e.g., a patient) are described. The disclosed methods comprise the use of library preparation steps that prevent strand displacement by polymerases and/or block resynthesis activity by polymerases during end repair and end modification to prevent incorporation of unmethylated cytosines in place of methylated cytosines. The disclosed methods enable more accurate determinations of methylation status based on sequence read data, and may improve the ability to detect hypomethylation biomarkers associated with disease, e.g., cancer.

In some instances, for example, methods are described that comprise extracting one or more nucleic acid fragnments (e.g., DNA fragments) from a sample obtained from a subject; performing at least one of: a first end repair reaction to repair fragmentation damage to one or more nucleic acid fragment ends, wherein the first end repair reaction comprises the use of a non-strand-displacing polymerase; or a second end repair reaction to repair fragmentation damage to one or more nucleic acid fragment ends, wherein the second end repair reaction comprises the use of a chain termination mechanism; performing at least one of: a tailing reaction to add an overhanging polynucleotide strand to the one or more end-repaired nucleic acid fragments, wherein the tailing reaction comprises the use of a single deoxynucleotide triphosphate (dNTP); or a nick/gap repair reaction to fill in single-stranded nicks or gaps in the one or more end-repaired nucleic acid fragments, wherein the nick/gap repair reaction comprises the use of a ligase to generate a plurality of modified nucleic acid fragments; and performing methylation analysis of the plurality of modified nucleic acid fragments.

In some instances, the methylation analysis comprises performing a cytosine conversion reaction on the plurality of modified nucleic acid fragments to generate a plurality of converted nucleic acid fragments.

In some instances, the methylation analysis comprises sequencing the plurality of converted nucleic acid fragments to generate a plurality of sequence reads. In some instances, the method further comprises performing a sequence read analysis based on the plurality of sequence reads to determine a methylation signature of the subject.

In some instances, the one or more nucleic acid fragments comprise one or more DNA fragments. In some instances, the non-strand-displacing polymerase comprises a non-strand-displacing DNA polymerase. In some instances, the ligase comprises a DNA ligase.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Mitigating Methylation Bias When Preparing DNA Sequencing Libraries

In mammalian genomes, methylation is primarily observed in genomic regions high in cytosine and guanine content comprising CpG islands. Methylation in these regions typically occurs in both strands of the dsDNA structure, i.e., if a cytosine on one strand of the duplex is methylated within a CpG locus, typically a nearby cytosine within a CpG locus on the opposite strand will also be methylated. If there is a nick or a gap on one strand of a dsDNA molecule that contains CpG island regions, the strand displacement and subsequent resynthesis of the DNA strand that may occur when performing conventional end repair and nick/gap repair reactions will erroneously incorporate unmethylated cytosines into the strand. For cytosines that were originally methylated, that methylation information is lost during the end repair and nick/gap repair process. This library preparation-based artifact is referred to as methylation bias (M-bias). M-bias results in loss of methylation status information and increased noise in sequence read-based methylation analysis, especially when attempting to analyze regions of the genome that, e.g., become hypomethylated during development or in disease states such as cancer. Reducing or eliminating M-bias can improve signal-to-noise ratios to more sensitive detection of methylation status in, for example, cancerous samples exhibiting lower levels of tumor fraction. Sensitive detection of regions of the genome that become hypomethylated in cancer is important for cancer detection and tissue of origin determination.

The novel end repair and nick/gap repair methods disclosed herein minimize M-bias when preparing dsDNA libraries intended for NGS. These methods are particularly relevant for NGS assays that query the methylation state of dsDNA molecules. Suppressing M-bias using the methods described herein are of key importance in achieving a high degree of sensitivity in, for example, cancer detection (especially early stage) and minimum residual disease (MRD) assays.

Use of single stranded library preparation strategies is a typical M-bias mitigation approach since there are no overhangs in single-stranded DNA molecules. However, there are disadvantages associated with single stranded library preparation, for example, lower molecular recovery and overall yields. Additionally, single-stranded library preparation requires denaturing duplexed DNA early on in the library preparation process, thereby losing the capability to use downstream double-stranded error suppression techniques that are available for double-stranded library preparation and sequencing. In some instances, the disclosed methods, e.g., the use of Taq DNA ligase in combination with a non-strand displacing polymerase for performing nick/gap repair as a very early step, could also be used for single-strand library preparation.

The disclosed methods have the advantage of lowering or eliminating methylation bias in double-stranded DNA library preparation. In contrast to single-stranded library preparation protocols, double-stranded library preparation is more efficient, thereby resulting in higher molecular recovery and increased overall library yields. The advantages of mitigating M-bias in a double-stranded library preparation workflow include higher molecular recovery and yields while preserving double strand error suppression for both hypermethylated and hypomethylated biomarkers. The disclosed methods result in better signal-to-noise in sequence read data for hypomethylated cancer biomarkers and enable better sensitivity for detection of methylation status at low tumor fraction where the amount of available circulating tumor DNA (ctDNA) in the sample is typically quite low (e.g., less than 1%).

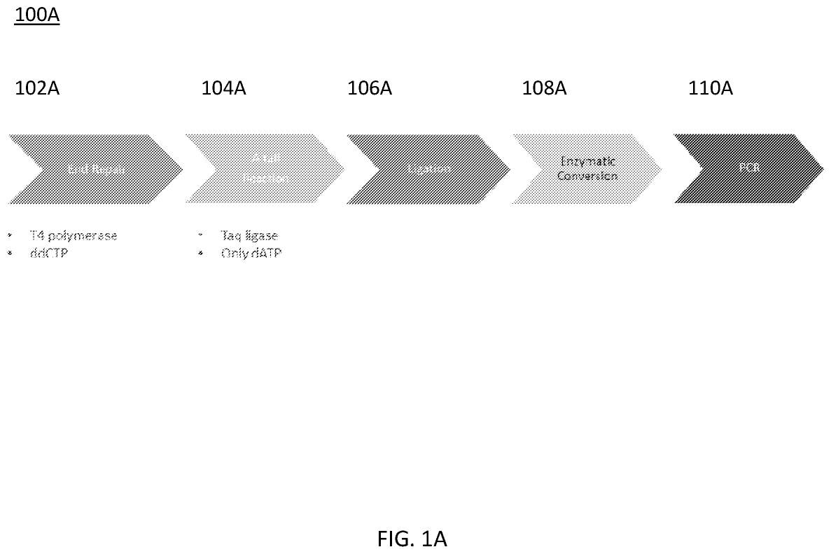

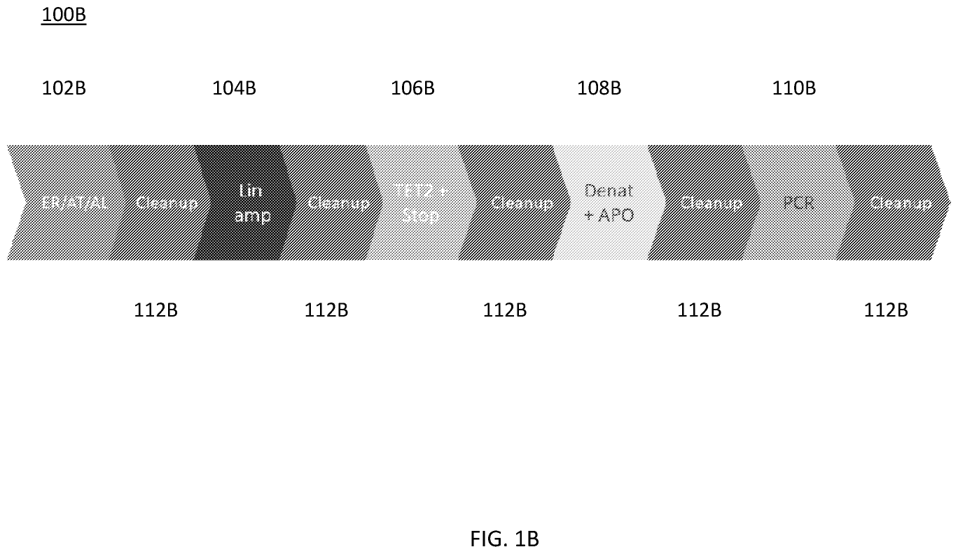

FIG. 1A provides a non-limiting example of a process 100A for preparing nucleic acid (e.g., DNA) sequencing libraries for methylation analysis, according to some implementations of the disclosed methods.

At step 102A in FIG. 1A, an end repair reaction is performed to convert fragmented nucleic acid molecules into blunt-end nucleic acid molecules containing 5′-phosphate and 3′-hydroxyl groups. The 5′→3′ polymerase activity of DNA polymerase(s) used in the reaction mixture fills in 5′ overhangs, while the 3′→5′ exonuclease activity of the DNA polymerase(s) removes 3′ overhangs. In some instances, the fragmented nucleic acid molecules may be, e.g., naturally-occurring fragmented DNA, such as circulating tumor DNA (ctDNA) which is typically less than 300 bps in length. In some instances, the fragmented nucleic acid molecules may be, e.g., DNA that has been extracted from a tissue sample and fragmented using mechanical methods (e.g., sonication) and/or enzymatic methods (e.g., using a fragmentase and/or restriction enzymes).

At step 104A in FIG. 1A, an A-tail reaction (e.g., an enzymatic reaction for adding a non-templated nucleotide to the 3′ end of a blunt, double-stranded DNA molecule) is performed.

At step 106A in FIG. 1A, a ligation reaction is performed, e.g., to ligate sequencing adapters onto the ends of the end repaired and/or tailed DNA fragments.

At step 108A in FIG. 1A, an enzymatic cytosine conversion reaction is performed, e.g., to convert non-methylated cytosine to uracil. Many DNA sequencing-based methylation analysis workflows use either a chemical conversion reaction (e.g., bisulfite) or an enzymatic conversion to convert unmethylated cytosines into uracils. After sequencing and mapping of sequence reads to a reference genome, these uracil residues will manifest as C→T substitution mutations, which can then be used to infer the methylation status of that locus of the genome. However, there are also sequencing technologies that can be used to detect methyl cytosine directly without the need for an additional conversion step. The methods described in this disclosure can be used to reduce M-bias regardless of the specific sequencing technology utilized.

At step 110A in FIG. 1A, a nucleic acid amplification reaction (e.g., a PCR reaction) is performed, e.g., to create additional copies of the end repaired, tailed, and/or adapter ligated DNA fragments following conversion of non-methylated cytosine to uracil.

FIG. 1B provides a non-limiting example of another process 100B for preparing nucleic acid (e.g., DNA) sequencing libraries for methylation analysis, according to some implementations of the disclosed methods.

At step 102B in FIG. 1B, end repair (ER), A-tailing (AT), and adapter ligation (AL) reactions are performed. An end repair reaction is performed to convert fragmented nucleic acid molecules into blunt-end nucleic acid molecules containing 5′-phosphate and 3′-hydroxyl groups. The 5′→3′ polymerase activity of DNA polymerase(s) used in the reaction mixture fills in 5′ overhangs, while the 3′→5′ exonuclease activity of the DNA polymerase(s) removes 3′ overhangs. In some instances, the fragmented nucleic acid molecules may be, e.g., naturally-occurring fragmented DNA, such as circulating tumor DNA (ctDNA) which is typically less than 300 bps in length. In some instances, the fragmented nucleic acid molecules may be, e.g., DNA that has been extracted from a tissue sample and fragmented using mechanical methods (e.g., sonication) and/or enzymatic methods (e.g., using a fragmentase and/or restriction enzymes). An A-tail reaction (e.g., an enzymatic reaction for adding a non-templated nucleotide to the 3′ end of a blunt, double-stranded DNA molecule) is also performed. Finally, an adapter ligation reaction is performed, e.g., to ligate sequencing adapters onto the ends of the end repaired and/or tailed DNA fragments.

At step 104B in FIG. 1B, a linear amplification reaction is performed. Linear amplification is a method for synthesizing single-stranded DNA from either single-stranded DNA or one strand of a double-stranded DNA molecule (see, e.g., Chakravarti et al. (2008), “Formation of Template-Switching Artifacts by Linear Amplification”, J Biomol Tech. 19(3):184-188). To perform linear amplification, molecules of a single primer DNA are extended by multiple rounds of DNA synthesis at high temperature using thermostable DNA polymerases (e.g., Tth DNA polymerase, Vent DNA polymerase, etc.).

At step 106B in FIG. 1B, a first enzymatic reaction is performed to protect methylated cytosines from deamination during cytosine conversion reactions used, e.g., to convert non-methylated cytosine to uracil. For example, Tet methylcytosine dioxygenase 2 (TET2) can be used, optionally in combination with an oxidation enhancer, to catalyze the oxidization of 5mC to 5hmC, then to 5-formylcytosine (5fC), and finally to 5caC (see, e.g., Vaisvila et al. (2021), “Enzymatic Methyl Sequencing Detects DNA Methylation at Single-Base Resolution from Picograms of DNA”, Genome Research 31:1280-1289), and thereby protect them from downstream deamination by APOBEC. In some instances, the oxidation reaction can be halted by addition of an appropriate stop reagent.

At step 108B in FIG. 1B, a second enzymatic reaction is performed to convert non-methylated cytosine to uracil. For example, apolipoprotein B mRNA-editing enzyme, catalytic polypeptide (APOBEC) can be used to selectively deaminate cytosines (e.g., under nucleic acid denaturing conditions) and convert them to uracil residues. After sequencing and mapping of sequence reads to a reference genome, these uracil residues will manifest as C→T substitution mutations, which can then be used to infer the methylation status of that locus of the genome.

At step 110B in FIG. 1B, a nucleic acid amplification reaction (e.g., a PCR reaction) is performed, e.g., to create additional copies of the end repaired, tailed, and/or adapter ligated DNA fragments following conversion of non-methylated cytosine to uracil.

In some instances, one or more cleanup steps (steps 112B in FIG. 1B) may be performed following one or more of steps 102B, 104B, 106B, 108B, and 110B. In some instances, as illustrated in FIG. 1B, cleanup steps (steps 112B) may be performed after each of steps 102B, 104B, 106B, 108B, and 110B. Examples of suitable nucleic acid sample cleanup methods to, for example, remove enzymes or other proteins, perform buffer exchange, etc., include, but are not limited to, phenol/chloroform extraction, ethanol precipitation, lithium chloride precipitation, agarose gel electrophoresis, anion exchange chromatography, magnetic bead capture, etc., or any combination thereof.

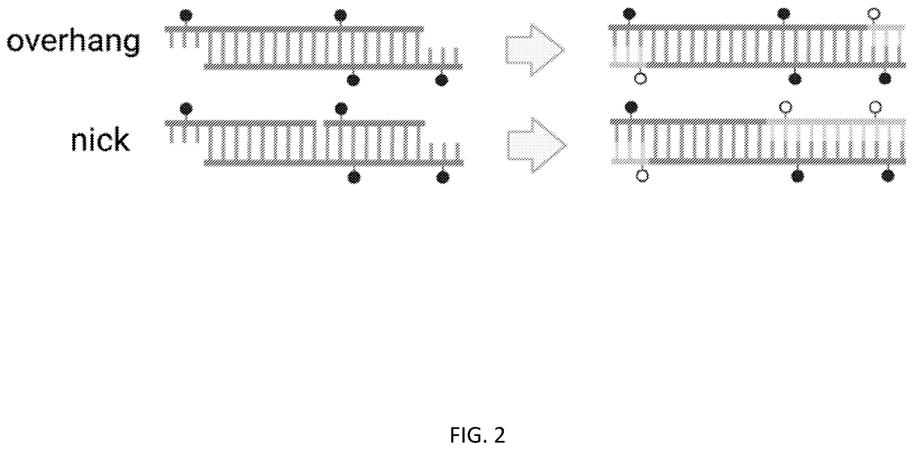

FIG. 2 provides a non-limiting schematic illustration of end repair and nick/gap repair reactions performed during the preparation of DNA sequencing libraries. As can be seen in the figures, end repair fills in 5′ overhangs (upper panel) and can result in replacement of what may have been methylated cytosines in CpG sites in the original double-stranded molecule with non-methylated cytosines in the repaired ends. Nick/gap repair can result in lengthy sections of incorporated bases (lower panel) due to strand displacement and resynthesis by the DNA polymerase used for end repair, again resulting in replacement of methylated cytosines in CpG sites with non-methylated cytosines.

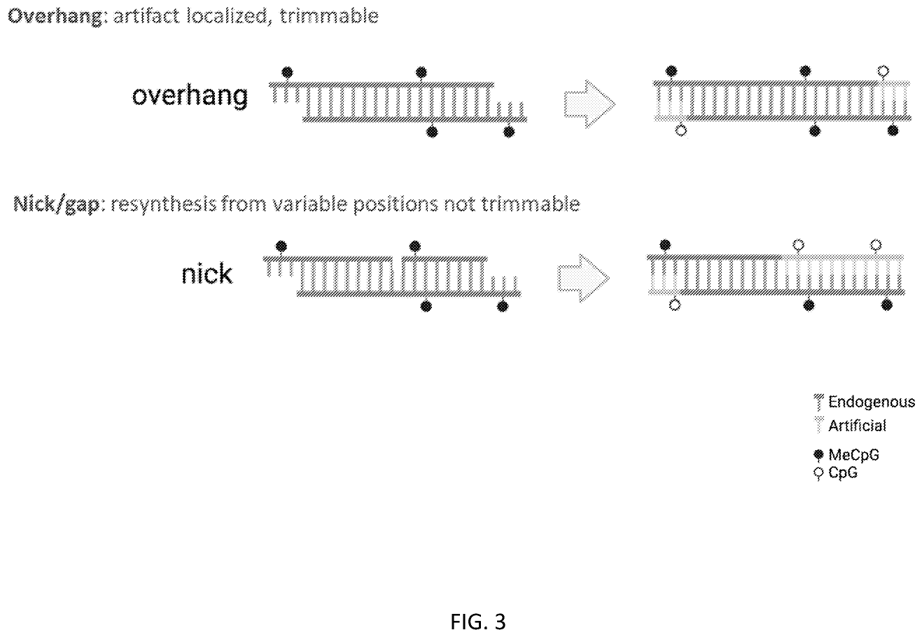

FIG. 3 provides another non-limiting schematic illustration of end repair and nick/gap repair reactions performed during the preparation of DNA sequencing libraries, and the methylation artifacts that can arise as a result. As indicated in the upper panel, methylation bias resulting from replacement of original methylated cytosines with non-methylated cytosines during end repair can be mitigated to some extent by computational trimming (e.g., excluding the data for a specified number of bases from methylation status calling) due to the localization of artifacts near the fragment ends. Nick/gap repair, on the other hand, can lead to methylation artifacts that cannot be mitigated by computational trimming because they arise from resynthesis of the displaced strands.

The disclosed methods reduce or eliminate M-bias when preparing sequencing libraries by implementing one or more of the following steps either individually or in combination:
Performing nick/gap repair using a DNA ligase, e.g., Taq ligase. Alternative examples of ligases that may be used for performing nick/gap repair include, but are not limited to, T4 ligase, 9°NÔ DNA ligase, T3 DNA ligase, E. coli ligase, etc., or any combination thereof.
Performing end repair using a polymerase enzyme that lacks strand displacement activity. Polymerases that lack strand displacement activity lack the ability to displace downstream duplex DNA strands encountered during synthesis. There are a variety of DNA polymerases available that have varying degrees of strand displacement activity. Examples of DNA polymerases that lack strand displacement activity include, but are not limited to, T4 DNA polymerase, Klentaq, etc.
Adding 2′,3′-dideoxycytidine 5′-triphosphate (ddCTP) (100% or dilute) to the end repair reaction mix. Adding 2′,3′-dideoxycytidine 5′-triphosphate (ddCTP) (100% or dilute) to the end repair reaction mix instead of, or in addition to, 2′-deoxycytidine 5′-triphosphate (dCTP) to eliminate all 5′ overhang repaired molecules, or to limit the amount of repair allowed in each molecule. Alternatively, 2′,3′-dideoxythymidine 5′-triphosphate (ddTTP), 2′,3′-dideoxy guanosine 5′-triphosphate (ddGTP), 2′,3′-dideoxyadenosine 5′-triphosphate (ddATP) (or any combination of ddCTP, ddTTP, ddGTP, and ddATP) can be used. In some instance, the concentration of ddNTP (or other chain terminating nucleotides) used may be varied, as different polymerases have different tolerances to non-natural nucleotides. In some instances, other chain termination moieties or chain termination mechanisms may be used as well. For example, chain termination during the end repair (blunting) reaction may also be achieved by omitting a certain dNTP (e.g., dATP, dTTP, dCTP, or dGTP) from the reaction mixture (or including only a limiting amount of a certain dNTP). In some instances, a limiting amount of a specified dNTP may correspond to a concentration that is about 1/200^{th}, 1/100^{th}, 1/90^{th}, 1/80^{th}, 1/70^{th}, 1/60^{th}, 1/50^{th}, 1/40^{th}, 1/30^{th}, 1/20^{th}, or 1/10^{th} the concentration of the other deoxynucleotide triphosphates in the reaction mixture. In some instances, a ratio of ddNTP concentration to a corresponding dNTP concentration used to perform the end repair reaction may be less than 20×, 30×, 40×, 50×, or 60×, 70×, 80×, 100×, 120×, or 140×. In some instances, noncanonical or modified nucleotides may be used in the end repair (blunting) process to flag regions where end repair occurs. Examples of modified nucleotides that may be used include, but are not limited to, methylated cytidine (e.g., 5-methyl-dCTP and 5-hydroxy-methyl-dCTP), deoxy uracil, and/or oxoguanine.
Perform a tailing using only a single deoxynucleotide triphosphate (dNTP), e.g., 2′-deoxyadenosine 5′-triphosphate (dATP). The tailing reaction may also be a source of unwanted strand displacement. In the disclosed methods, using only 2′-deoxyadenosine 5′-triphosphate (dATP) during the tailing reaction limits strand resynthesis. However, omission of any of the four dNTPs, or omitting the tailing reaction step completely may also be used to limit strand resynthesis. If a tailing reaction (e.g., A-tailing reaction) is completely omitted, adapters may be appended via blunt ligation. In some instances, the tailing reaction may be performed using 2′-deoxyadenosine 5′-triphosphate (dATP), 2′-deoxythymidine 5′-triphosphate (dTTP), 2′-deoxycytidine 5′-triphosphate (dCTP), 2′-deoxyguanosine 5′-triphosphate (dGTP), or any combination thereof. In some instances, the tailing reaction may be performed using a single deoxynucleotide triphosphate, e.g., dATP, dTTP, dCTP, or dGTP. Examples of DNA polymerases that may be used for performing the tailing reaction include, but are not limited to, T4 DNA polymerase, T4 Polynucleotide Kinase Reaction (T4 PNK), Taq DNA polymerase, Klenow Fragment (3′→5′ exo-), etc. In some instances, alternative polymerases (e.g., Sulfolobus DNA polymerase IV) that lack strand displacement activity but that have the ability to append a 3′ dA may be used to perform a tailing reaction, e.g., an A-tailing reaction.

In some instances, 5-methyldeoxycytidine 5′-triphosphate (5-methyl-dCTP) or another modified nucleotide may be added as part of the reaction mixture used for any of the four steps listed above to facilitate detection of repaired bases. Additional examples of modified nucleotides that may be used include, but are not limited to, 5-hydroxymethyldeoxycytidine 5′-triphosphate, deoxyuradine 5′-triphosphosphate, 8-oxo-2-deoxyguanosine 5′-triphosphate, or any combination thereof.

In some instances, the disclosed methods may comprise performing one or more of the four steps listed above during library preparation in order to prevent or reduce M-bias during conversion and sequencing. In some instances, the disclosed methods may comprise performing one, two, three, or all four of: (i) a first end repair reaction to repair fragmentation damage to one or more DNA fragment ends, wherein the first end repair reaction comprises the use of a non-strand-displacing DNA polymerase; (ii) a second end repair reaction to repair fragmentation damage to one or more DNA fragment ends, wherein the second end repair reaction comprises the use of a chain termination mechanism; (iii) a tailing reaction to add an overhanging polynucleotide strand to the one or more end-repaired DNA fragments, wherein the tailing reaction comprises the use of a single deoxynucleotide triphosphate (dNTP); and/or (iv) a nick/gap repair reaction to fill in single-stranded nicks or gaps in the one or more end-repaired DNA fragments, wherein the nick/gap repair reaction comprises the use of a DNA ligase, as part of preparing a DNA sequencing library.

In some instances, the disclosed methods may comprise performing at least one of: (i) a first end repair reaction to repair fragmentation damage to one or more DNA fragment ends, wherein the first end repair reaction comprises the use of a non-strand-displacing DNA polymerase; or (ii) a second end repair reaction to repair fragmentation damage to one or more DNA fragment ends, wherein the second end repair reaction comprises the use of a chain termination mechanism; and performing at least one of: (iii) a tailing reaction to add an overhanging polynucleotide strand to the one or more end-repaired DNA fragments, wherein the tailing reaction comprises the use of a single deoxynucleotide triphosphate (dNTP); or (iv) a nick/gap repair reaction to fill in single-stranded nicks or gaps in the one or more end-repaired DNA fragments, wherein the nick/gap repair reaction comprises the use of a DNA ligase.

In some instances, the disclosed methods may comprise performing both the first end repair reaction and the second end repair reaction. In some instances, the disclosed methods may comprise performing both the tailing reaction comprising the use of a single dNTP and the nick/gap repair reaction comprising the use of a DNA ligase.

In some instances, the disclosed library preparation methods may be performed on double-stranded DNA fragments. In some instances, one or more of the four steps outlined above may be performed on single-stranded DNA fragments.

In some instances, as described in more detail below, the disclosed methods may comprise performing a methylation analysis on nucleic acid molecules derived from the plurality of modified DNA fragments in the resulting sequencing library. In some instances, the methylation analysis comprises a restriction enzyme-based, affinity enrichment-based, bisulfite conversion-based, or enzymatic conversion-based methylation sequencing method.

In some instances, the disclosed methods may further comprise performing a ligation step to ligate one or more adapters to the plurality of modified DNA fragments. In some instances, the one or more adapters comprise one or more adapters comprising an overhanging poly-T sequence. In some instances, the one or more adapters comprise one or more sequencing adapters, for example, one or more methylated stubby adapters, flow cell adapters, read 1 sequencing adapters, read 2 sequencing adapters, or any combination thereof. In some instances, the one or more adapters comprise one or more barcodes. In some instances, the disclosed methods may further comprise performing a ligation step to add a one or more barcodes to the plurality of modified DNA fragments. In some instances, the one or more barcodes may comprise, for example, a library index, a sample barcode, a cell barcode, a target-specific barcode, a DNA fragment barcode, a unique molecular index, or any combination thereof.

In some instances, the disclosed methods may comprise performing a cytosine conversion reaction on the plurality of modified DNA fragments to generate a plurality of converted DNA fragments. In some instances, the cytosine conversion reaction is used to convert non-methylated cytosine to uracil. In some instances, the cytosine conversion reaction comprises a chemical conversion reaction, e.g., a bisulfite conversion reaction. In some instances, the cytosine conversion reaction comprises an enzymatic conversion reaction, e.g., comprising the use of a tet methylcytosine dioxygenase 2 (TET2) enzyme to oxidize 5-methyl-cytosine (5mC) or 5-hydroxymethyl-cytosine (5hmC) to 5-carboxycytosine (5caC). In some instances, the enzymatic conversion reaction may further comprise the use of a combination of TET2 and T4 β-glucosyltransferase (T4-βGT) enzymes to convert 5-methyl-cytosine (5mC) or 5-hydroxymethyl-cytosine (5hmC) to 5-(D-glucosyl)oxymethyl-cytosine. In some instances, the enzymatic conversion reaction may comprise the use of an Apolipoprotein B mRNA Editing Catalytic Polypeptide-like (APOBEC) enzyme.

In some instances, the disclosed methods may further comprise performing a nucleic acid amplification reaction to generate amplified nucleic acid molecules that are derived from the plurality of modified DNA fragments. In some instances, for example, the nucleic acid amplification reaction may comprise a polymerase chain reaction (PCR). In some instances, the nucleic acid amplification reaction may comprise a rolling circle amplification (RCA) reaction.

In some instances, the disclosed methods may further comprise capturing a subset of nucleic acid molecules from the amplified nucleic acid molecules and performing nucleic acid sequencing and/or methylation analysis on the subset.

In some instances, the methylation analysis may comprise sequencing the plurality of converted DNA fragments to generate a plurality of sequence reads. In some instances, the disclosed methods may further comprise performing a sequence read analysis based on the plurality of sequence reads to determine a methylation signature of the subject.

In some instances, sequence read data obtained by sequencing nucleic acid molecules derived from the plurality of modified DNA fragments may exhibit reduced methylation bias compared to that obtained by sequencing a conventionally-prepared DNA sequencing library. For example, in some instances, the reduction in methylation bias may be greater than 5%, 10%, 15%, 20%, 25%, 30%, or 35% as measured by the SD methyl position bias metric described elsewhere herein.

In some instances, the nucleic acid sequencing and/or methylation analysis is performed using a next-generation sequencer. In some instances, the nucleic acid sequencing and/or methylation analysis may comprise using the next-generation sequencer to perform targeted sequencing. In some instances, the nucleic acid sequencing and/or methylation analysis may comprise using the next-generation sequencing to perform whole genome sequencing.

### Quantification of Methylation Bias

The extent to which these modified library preparation steps reduce or eliminate M-bias when preparing DNA libraries for sequencing can be quantified in a variety of ways. For example, one can quantify how methylation status varies across sequence read base position (the location of a given base within a given sequence read) for sequence reads aligned to all loci genome-wide based on a reference genome or a matched normal reference genome.

A non-limiting example of a process for analyzing different sources of methylation bias may include: (i) looking at methylation in a control region set, e.g., a set of genomic regions that show high levels of methylation in a wide variety of samples, including both cancer samples and healthy samples, (ii) analyzing the trend for methylation data (e.g., average methylation fraction (AMF) data) versus sequence read base position at the 5′ end of the DNA fragment; the level typically starts high and then shows an approximately linear decrease, and (iii) analyzing the methylation versus sequence read base position data using, for example, a regression model to determine the slope of the approximately linear decrease (termed the “slip rate”), which can be caused by artifacts introduced by the strand displacement activity of the polymerase at nicks and gaps. Near the 3′ end of the DNA fragment, particularly for Read 2 data, one can also see a stronger reduction in methylation levels that can be due to end repair. By applying a threshold (e.g., an AMF threshold) based on the linear regression model described above, one can determine, for example, the number of bases affected by end repair (or “end repair bases”) as the number of bases on the 3′ end of the fragment that are below the AMF methylation threshold.

Other characteristics of the pattern of M-bias can also be determined using these quantification methods, for example: the level of methylation that is observed at the 5′ end of each sequence read (“baseline level”), positional biases observed in other regions of the genome, methylation biases observed for under different sample types or under different laboratory (e.g., library preparation) conditions. The quantification methods can also be used to monitor other potential sources of artifact as a quality control measure.

### Methylation Analysis for Detection and Diagnosis of Disease, Etc.

As noted above, in some instances, the disclosed methods may further comprise performing a methylation analysis. In some instances, the methylation analysis may comprise performing a sequence read analysis based on a plurality of sequence reads obtained by sequencing a DNA library prepared using one or more of the four steps listed above to determine a methylation signature of a subject from which a DNA sample was extracted. For example, in some instances, determining a methylation signature may comprise determining a methylation status for each of one or more genomic loci based on the sequence read data for the plurality of sequence reads.

In some instances, the disclosed methods may further comprise screening, detecting, diagnosing, and/or confirming a diagnosis of, or monitoring disease in the subject based on sequence read data for the plurality of sequence reads. In some instances, the screening, detecting, diagnosing, confirming a diagnosis, or monitoring of disease may be performed with improved accuracy due to a reduction in methylation bias in the sequence read data compared to that obtained by sequencing a conventionally-prepared DNA sequencing library.

In some instances, the disclosed methods may further comprise detecting minimum residual disease in the subject based on sequence read data for a plurality of sequence reads obtained by sequencing a DNA sequencing library prepared using the methods disclosed herein. In some instances, for example, the disease may be cancer. In some instances, the methylation analysis performed using a DNA sequencing library prepared using the methods disclosed herein may be used to detect hypomethylated or hypermethylated genomic regions in cancer patients.

DNA sequencing is not the only method that may be used to quantify cytosine methylation in genomic DNA. The disclosed methods may be used to reduce or eliminate M-bias in DNA libraries for which cytosine methylation is quantified using, e.g., microarrays or other methylation analysis methods.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for preparing DNA sequencing libraries, performing nucleic acid sequencing, and/or performing a methylation analysis may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for preparing DNA sequencing libraries, performing nucleic acid sequencing, and/or performing a methylation analysis may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for preparing DNA sequencing libraries, performing nucleic acid sequencing, and/or performing a methylation analysis may be used to select a subject (e.g., a patient) for a clinical trial based on, e.g., the methylation status determined for one or more gene loci (or methylation signature) for the subject. In some instances, patient selection for clinical trials based on, e.g., determination of a specific methylation signature, may accelerate the development of targeted therapies and improve the healthcare outcomes for treatment decisions.

In some instances, the disclosed methods for preparing DNA sequencing libraries, performing nucleic acid sequencing, and/or performing a methylation analysis may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for preparing DNA sequencing libraries, performing nucleic acid sequencing, and/or performing a methylation analysis may be used in treating a disease (e.g., a cancer) in a subject. For example, in response to determining a methylation status of one or more genomic loci in sample from the subject using any of the methods disclosed herein, an effective amount of an anti-cancer therapy or anti-cancer treatment may be administered to the subject.

In some instances, the disclosed methods for preparing DNA sequencing libraries, performing nucleic acid sequencing, and/or performing a methylation analysis may be used for monitoring disease progression or recurrence (e.g., cancer or tumor progression or recurrence) in a subject. For example, in some instances, the methods may be used to determine a methylation signature in a first sample obtained from the subject at a first time point, and used to determine a methylation signature in a second sample obtained from the subject at a second time point, where comparison of the first determination of the methylation signature and the second determination of the methylation signature allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of a methylation status at one or more genomic loci and/or methylation signature for the subject.

In some instances, the methylation status at one or more genomic loci, or a methylation signature determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for preparing DNA sequencing libraries, performing nucleic acid sequencing, and/or performing a methylation analysis may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for preparing DNA sequencing libraries, performing nucleic acid sequencing, and/or performing a methylation analysis as part of a genomic profiling process (or inclusion of the output from the disclosed methods for preparing DNA sequencing libraries, performing nucleic acid sequencing, and/or performing a methylation analysis as part of the genomic profile of the subject) can improve the validity of, e.g., disease detection calls and treatment decisions, made on the basis of the genomic profile by, for example, independently confirming the methylation status at one or more genomic loci in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome.

For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof, methylated sequences or fragments thereof, or any combination thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent (e.g., a target capture probe) may comprise a binding agent such as a peptide or protein comprising a methyl-CpG binding domain (MDB) such that the target capture reagent (e.g., MBD-protein coupled beads) selectively binds nucleic acid sequences comprising methylated CpG sites. In some instances, the target nucleic acid is a nucleic acid sequence comprising one or more methylated CpG sites. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite-or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011;21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011;21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequencing—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for mitigating methylation bias during preparation of sequencing libraries for sequencing nucleic acid molecules (e.g., DNA molecules) extracted from a sample from a subject. The systems may comprise, e.g., one or more processors, and a memory unit communicatively coupled to the one or more processors and configured to store instructions that, when executed by the one or more processors, cause the system to: perform at least one of: a first end repair reaction to repair fragmentation damage to one or more DNA fragment ends, wherein the first end repair reaction comprises the use of a non-strand-displacing DNA polymerase; or a second end repair reaction to repair fragmentation damage to one or more DNA fragment ends, wherein the second end repair reaction comprises the use of a chain termination mechanism; perform at least one of: a tailing reaction to add an overhanging polynucleotide strand to the one or more end-repaired DNA fragments, wherein the tailing reaction comprises the use of a single deoxynucleotide triphosphate (dNTP); or a nick/gap repair reaction to fill in single-stranded nicks or gaps in the one or more end-repaired DNA fragments, wherein the nick/gap repair reaction comprises the use of a DNA ligase to generate a plurality of modified DNA fragments; and perform methylation analysis of the plurality of modified DNA fragments.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the systems disclosed herein may comprise: an extractor configured to extract a plurality of a plurality of nucleic acid molecules from a sample obtained from a subject; an automated library preparation device configured to execute one or more processes that when executed perform one or more of: a first end repair reaction to repair fragmentation damage to one or more ends of the nucleic acid molecules, wherein the first end repair reaction comprises the use of a non-strand-displacing DNA polymerase; or a second end repair reaction to repair fragmentation damage to the one or more ends, wherein the second end repair reaction comprises the use of a chain termination mechanism; performing at least one of: a tailing reaction to add an overhanging polynucleotide strand to the one or more end-repaired nucleic acid molecules, wherein the tailing reaction comprises the use of a single deoxynucleotide triphosphate (dNTP); or a nick/gap repair reaction to fill in single-stranded nicks or gaps in the one or more end-repaired nucleic acid molecules, wherein the nick/gap repair reaction comprises the use of a DNA ligase to generate a plurality of modified nucleic acid molecules; a sequencer configured to sequence the modified nucleic acid molecules and generate one or more sequence modified nucleic acid sequence reads representing the nucleic acid sequence of the modified nucleic acid molecule; and a computational analysis platform including one or more processors including a memory that stores one or more processes, the processes when executed configured to analyze the modified nucleic acid reads and identify one or more alterations or epigenetic signatures in the modified nucleic acid molecules.

Examples of suitable extractors include, but are not limited to, the KingFisher™ Flex Purification System (ThermoFisher Scientific, Waltham, MA), the QIAsymphony SP, QIAcubeHT, EZ1 Advanced XL, and EZ2 Connect instruments from Qiagen (Qiagen, Germantown, MD), the Swift™ Extract Automated Nucleic Acid Extraction System (ESCO Lifesciences Group, Horsham, PA), the HSM 2.0 and Maxprep™ Liquid Handler instruments from Promega (Promega, Madison, WI), and the MagNA Pure 96 Instrument (Roche Diagnostics Corp., Indianapolis, IN).

Examples of suitable automated library preparation devices include, but are not limited to, the ADAP STAR and NGS STAR instruments from Hamilton (Hamilton Company, Reno, NV), the Bravo NGS Workstation (Agilent Technologies, Santa Clara, CA), the Biomek Genomics Workstation (Beckman Coulter Life Sciences, Indianapolis, IN), and the MagicPrep™, DreamPrep®, and DreamPrep® Compact systems from Tecan (Männedorf, Switzerland).

In some instances, the disclosed systems may be used for preparing sequencing libraries and/or for performing sequencing of nucleic acid molecules (e.g., DNA molecules) extracted from any of a variety of samples as described herein (e.g., a tissue sample, biopsy sample, hematological sample, or liquid biopsy sample derived from the subject).

In some instances, the plurality of genomic loci (e.g., CpG sites) for which sequencing data is processed to determine a methylation state may comprise at least 1, 2, 3, 4, 5, 6, 7, 8, 9, 10, 20, 30, 40, 50, 60, 70, 80, 90, 100, 200, 300, 400, 500, 600, 700, 800, 900, 1000, or more than 1000 genomic loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a methylation state at one or more CpG loci, or a methylations signature for DNA extracted from a sample from a subject may be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

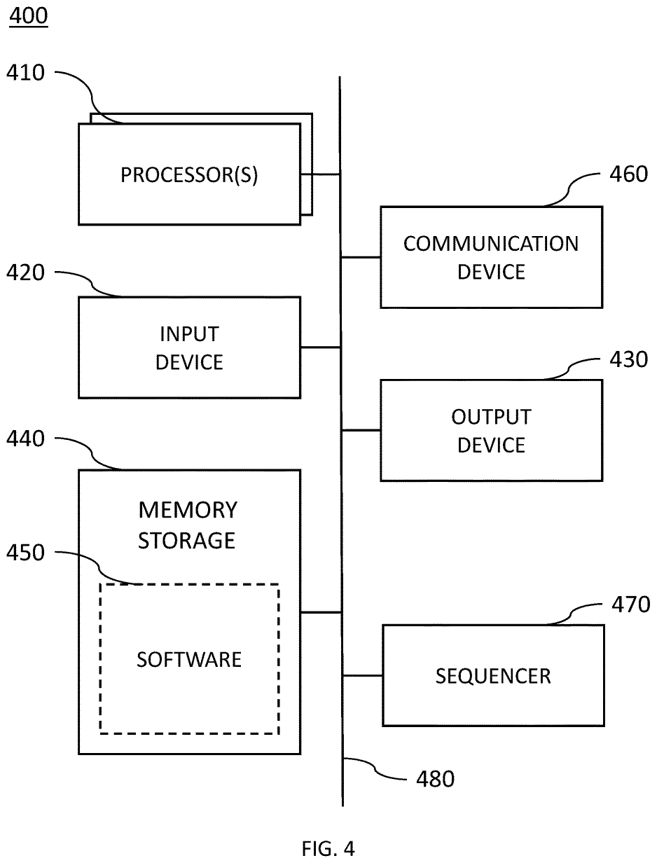

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Software 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

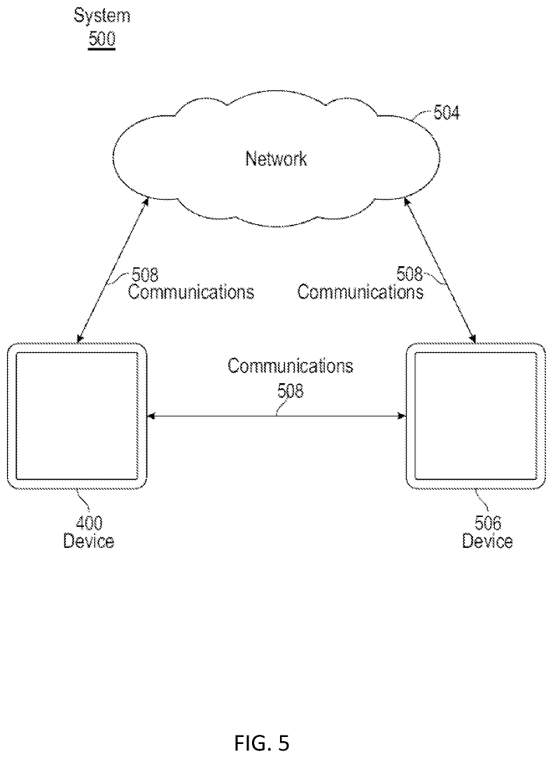

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e. g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### EXAMPLES

The following examples are included for illustrative purposes only and are not intended to limit the scope of the present disclosure.

### Example 1—Detection of Methylation Bias

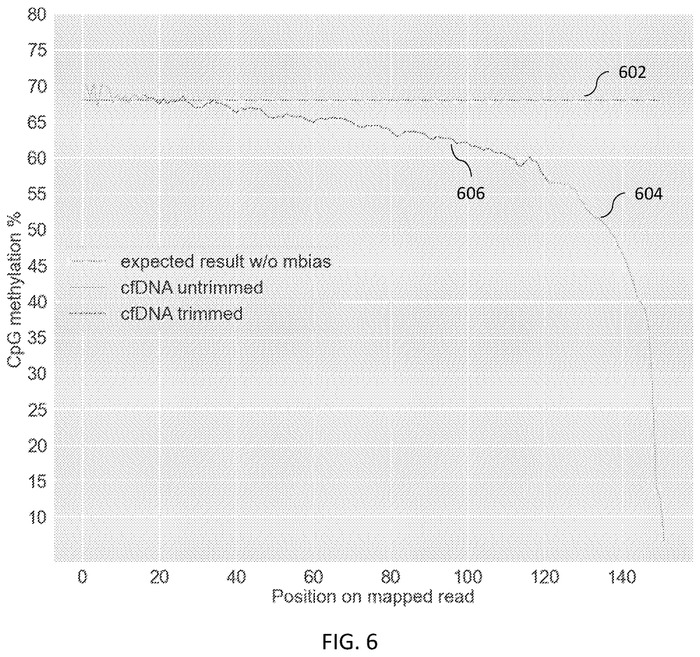

FIG. 6 provides a non-limiting example of data that illustrates the effect of methylation bias in sequence read data. Sequence read base position (the location of a given base within a given sequence read) should have no influence on the methylation state when that base is a cytosine base, i.e., if the methylation state of a given CpG site at a given genomic position is averaged across all sequence reads that cover that position (the number of sequence reads that have at least one base that aligns to a given position on the genome at a given location), the average value (or percent methylated) should be independent of sequence read position (FIG. 6, flat line 602). However, when the methylation state of a given CpG site is averaged over all sequence reads from a cell-free DNA (cfDNA) sample that cover that genomic position, a rapid decrease in the average value (or percent methylated) is observed (FIG. 6, curve 604), which is indicative of error introduced by the library preparation steps. In some instances, methylation bias may be partially corrected for analytically, e.g., by trimming the sequence read data to exclude data for those portions of a sequence read that exhibit the largest methylation bias (e.g., the last 20 nucleotides or so; see FIG. 6, curve 606—which corresponds to the dotted portion of curve 604). In some instances, methylation position bias can be quantified, e.g., by calculating the average and/or standard deviation of the observed methylation percentage (e.g., the standard deviation (SD) methylation position bias) across the length of a sequence read. For the example shown in FIG. 6, the standard deviation for methylation position bias was 0.038. Elimination of methylation bias would improve the ability to detect hypomethylation biomarkers in cancer by minimizing erroneously low background methylation levels in samples from healthy individuals.

### Example 2—Mitigation of Methylation Bias Using Modified Library Preparation Steps

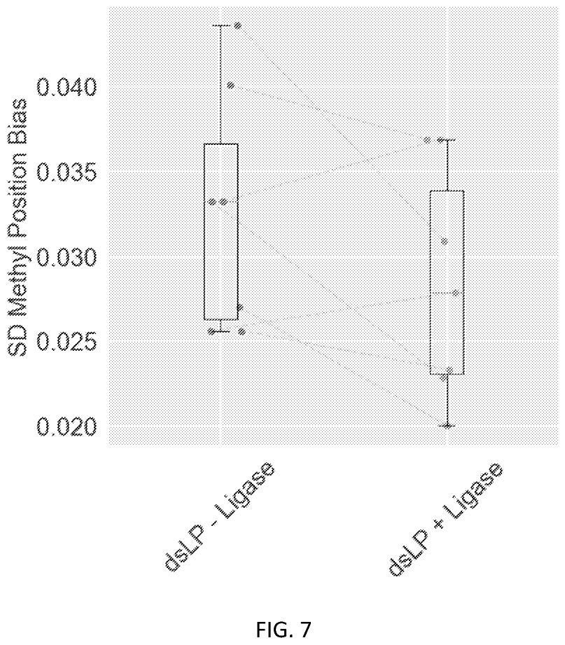

As discussed herein, modifying the reaction conditions used to perform end repair, nick and gap repair, and/or tailing during sequencing library preparation can be used to minimize or eliminate the methylation bias observed in sequence read data. FIG. 7 provides a non-limiting example of a box plot of data for observed methylation bias (quantified as standard deviation (SD) methylation position bias) with and without using Taq DNA ligase to perform nick or gap repair during library preparation. The dashed lines connect matched samples (i.e., the same samples that were tested with and without using Taq ligase). Again, methylation bias was quantified by calculating the standard deviation of the observed methylation percentage across the length of a sequence read. As can be seen, use of the Taq DNA ligase step resulted in lower overall methylation bias.

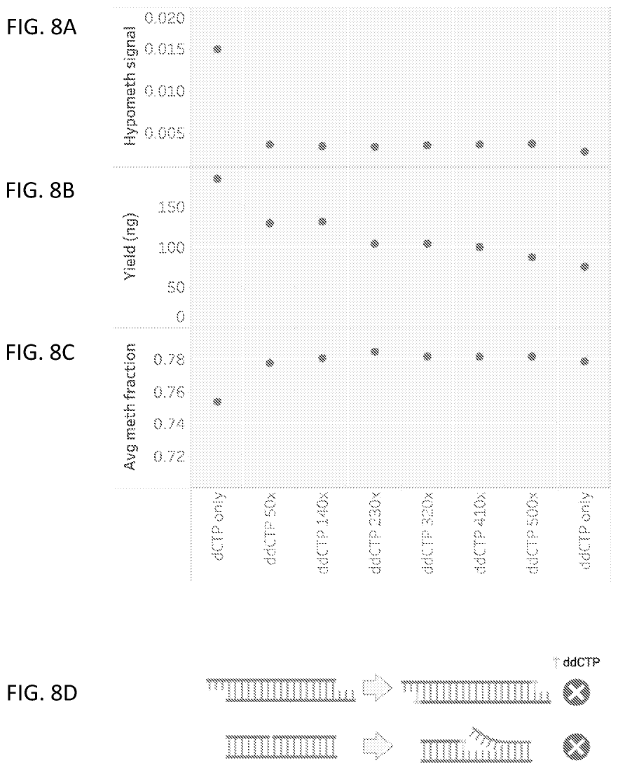

FIGS. 8A-C provide non-limiting examples of data for hypomethylation signal (FIG. 8A), yield (FIG. 8B), and average methylation fraction (FIG. 8C) for eight different end repair and nick/gap repair protocols that comprises the use of the Klenow fragment and mixtures of deoxycytidine triphosphate (dCTP) and dideoxycytidine triphosphate (ddCTP). Hypomethylation signal was calculated as the number of aligned sequence reads that are fully unmethylated divided by the total number of aligned sequence reads (for healthy/unaffected samples, the hypomethylation signal should be close to zero). As illustrated in FIG. 8D, inclusion of ddCTP in the end repair and nick/gap repair reaction partially excludes nucleic acid molecules (e.g., DNA molecules) comprising nicks or jagged ends (e.g., by blocking polymerase resynthesis activity) from participation in downstream library preparation steps and thus eliminates a potential source for methylation bias. The ddCTP blocks resynthesis of a strand of a duplex arising from, e.g., a nicked site, and thereby prevents long fill-ins resulting from resyntheses. The ddCTP is diluted to allow some repair to proceed to make the library preparation process more efficient. As result, downstream analytical programs are more effective at detecting and trimming sequence read data comprising localized 3′ repaired bases. By diluting the ddCTP, one can control how many artificial cytosines are incorporated into each repaired fragment. The more cytosines required to be repaired in a given fragment, the more likely it is to be excluded from the double-stranded ligation reaction. As shown in FIG. 8A, even at the lowest concentration of ddCTP tested (0.2 mM ddCTP, or 50× relative to the dCTP concentration (0.004 mM) used; ddCTP concentration was held constant at 0.2 mM and dCTP concentration was varied to test different concentration ratios), the presence of ddCTP resulted in a significant decrease of the hypomethylation signal in samples from healthy individuals. The yield data plotted in FIG. 8B shows titration of the overall reaction yield with ddCTP concentration, while the average methylation fraction data (FIG. 8C) levels off after the lowest concentration of ddCTP tested and indicates an average methylation fraction value of about 0.78 in healthy individuals. Adopting the least stringent condition (e.g., lowest ddCTP concentration) for performing library preparation means that fewer DNA molecules are sacrificed to achieve the observed reduction of methylation bias. In some instances, the optimal concentration of ddCTP concentration and/or ratio of ddCTP to dCTP in the reaction mixture may vary depending on which polymerase is used to perform end repair and/or nick/gap repair due, for example, to differences in their affinities for the polymerase and/or their relative incorporation efficiencies.

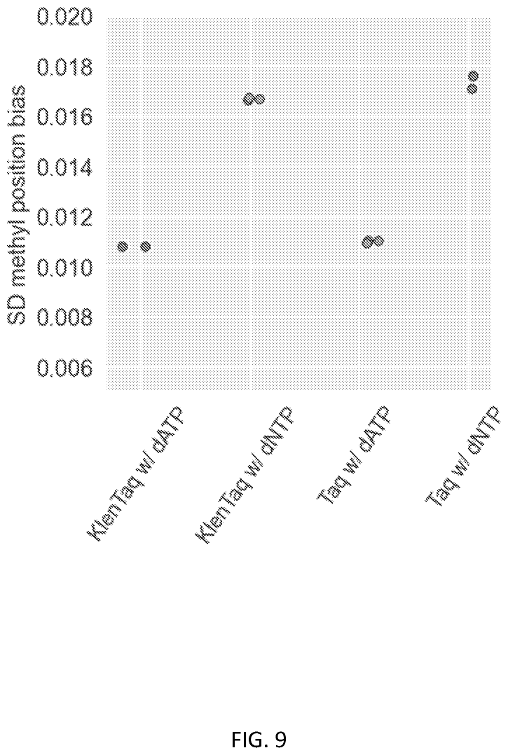

FIG. 9 provides a non-limiting example of data for methylation bias observed using four different tailing reaction protocols comprising the use of either the KlenTaq DNA polymerase or Taq DNA polymerase in combination with either a dNTP mixture or dATP only. Samples were blunted with T4 DNA polymerase, cleaned up, and then tailed with either KlenTaq DNA polymerase or Taq DNA polymerase in a buffer that contained either the mixture of dNTPs or dATP only. The KlenTaq DNA should not have strand displacement activity, while Taq DNA polymerase does. Again, methylation bias was quantified by calculating the standard deviation of the difference between the observed methylation percentage bias and the expected methylation percentage across the length of a sequence read. As can be seen in FIG. 9, use of dATP only (and either of the two polymerases) significantly lowered the observed methylation bias as compared to using a mixture of dNTPs (and either of the two polymerases) to perform the tailing reaction. These results also indicated that the displacement activity of the polymerase used to perform tailing had no effect.

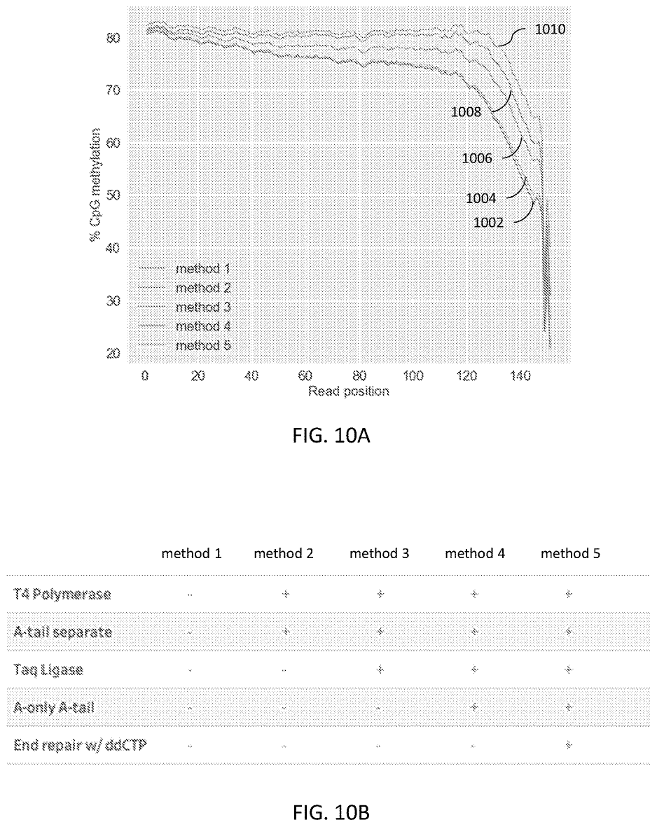

FIG. 10A provides examples of methylation bias data observed for the five different library preparation methods summarized in FIG. 10B. In order to test each method, representative libraries were prepared from unaffected/healthy cfDNA samples and from non-small cell lung cancer (NSCLC) cfDNA samples. The percent CpG methylation data (the percentage of CpG dinucleotide sites that comprise a methylated cytosine) plotted in FIG. 10A is for libraries prepared from unaffected cfDNA samples. As can be seen from curves 1002, 1004, 1006, 1008, and 1010 in FIG. 10A (corresponding to methods 1 (conventional library preparation), 2 (use of T4 polymerase for end repair and performing a separate A-tailing reaction during library preparation as a control), 3 (use of T4 polymerase for end repair, performing a separate A-tailing reaction, and using Taq ligase for nick/gap repair during library preparation), 4 (use of T4 polymerase for end repair, performing a separate A-tailing reaction using only dATP, and using Taq ligase for nick/gap repair during library preparation), and 5 (use of T4 polymerase and ddCTP for end repair, performing a separate A-tailing reaction using only dATP, and using Taq ligase for nick/gap repair during library preparation), respectively, as summarized in FIG. 10B), use of one or more of the modified end repair, nick or gap repair, and tailing reactions described herein resulted in significant decreases in observed methylation bias, which in turn enables improved sensitivity for detecting hypomethylated genomic regions based on the resulting sequence read data obtained for cancer patients. The A-tailing enzyme is usually included in an “all-in-one” reaction mixture comprising all of the end repair enzymes and nucleotides used to perform end repair, and is activated by changing the reaction temperature. Thus, in order to perform A tailing using only dATP, the A-tailing reaction was performed separately. As indicated by the data plotted in FIG. 10A, performing a separate A-tailing reaction by itself (using a mixture of nucleotides) didn't result in a reduction in methylation bias. The drop off in percent CpG methylation observed at around position 120 can be trimmed during data processing (analytical trimming) to further mitigate methylation bias.

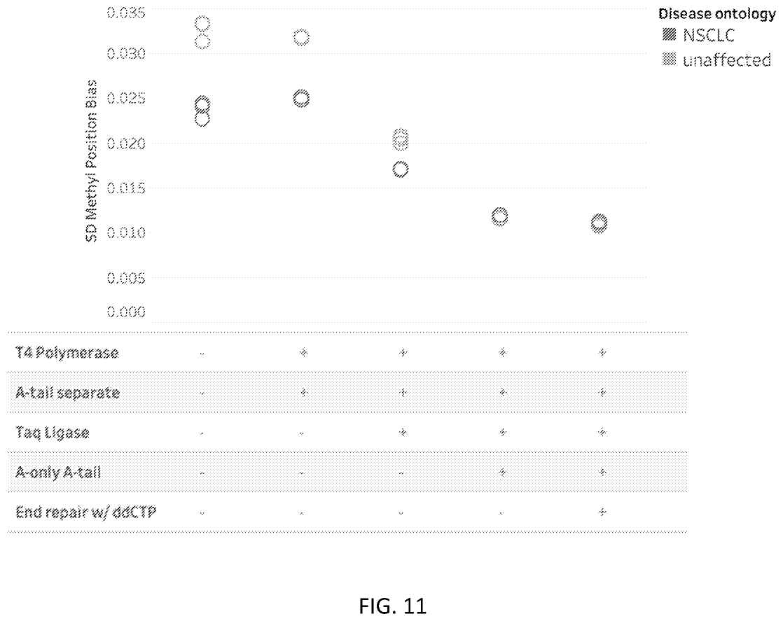

FIG. 11 provides non-limiting examples of methylation bias data (quantified as the standard deviation (SD) of the observed methylation bias) for sequence read data for DNA extracted from NSCLC and unaffected cfDNA samples using the five different library preparation methods summarized in the lower panel of the figure and described in reference to FIG. 10B. Again, the protocol comprising use of T4 polymerase and preforming a separate A-tailing reaction (for the reason described above) was included as a control. Incremental reductions in methylation bias were observed from the cumulative influence of using non-strand displacement enzymes to perform end repair, performing a separate A-tailing reaction using dATP only, and using Taq DNA ligase to perform nick/gap repair, with additional reductions achieved by also including the addition of ddCTP to the end repair reaction.

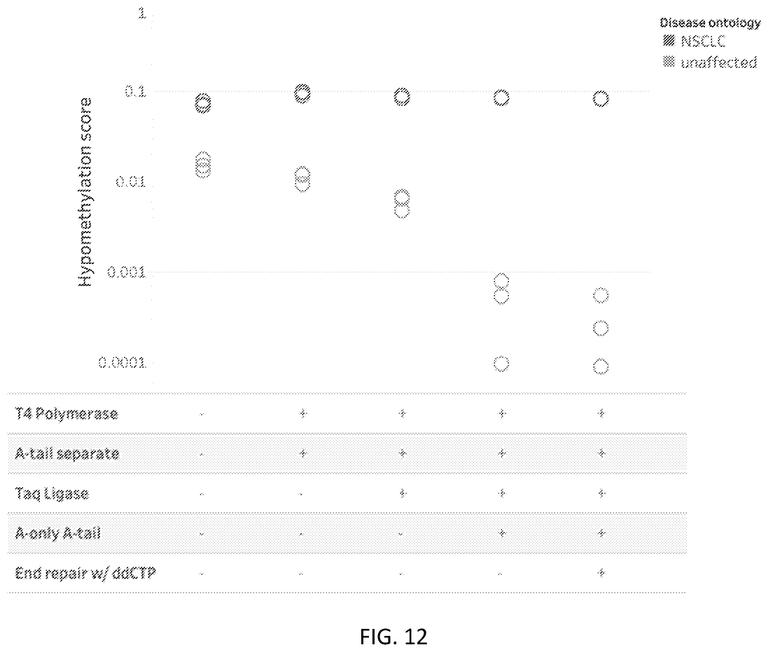

FIG. 12 provides non-limiting examples of hypomethylation scores calculated for the methylation bias data presented in FIG. 11, where a higher hypomethylation score is indicative of a sample exhibiting a higher level of hypomethylation. Hypomethylation scores may be calculated, e.g., as the proportion of all cancer-associated hypomethylation regions observed to be fully hypomethylated for a specific patient sample. If a patient exhibited complete hypomethylation at every single cancer-associated hypomethylation region in the set, their hypomethylation score would be 1. Note that the hypomethylation scores are plotted on a log scale axis. As can be seen in FIG. 12, the cumulative influence of using non-strand displacement enzymes for end repair, Taq DNA ligase for nick/gap repair, using only dATP in the A-tailing reaction, and/or the addition of ddCTP to the end repair reaction led to reduced hypomethylation in the sequence reads for unaffected samples (the healthy background was reduced by 98%), while the hypomethylation status for sequence reads from the NSCLC samples persists, as expected. Library preparation using T4 polymerase and a separate A-tailing reaction were included as controls, as described above. Greater differentiation between the methylation status called for diseased and healthy samples provides better assay sensitivity and more confident assay calls.

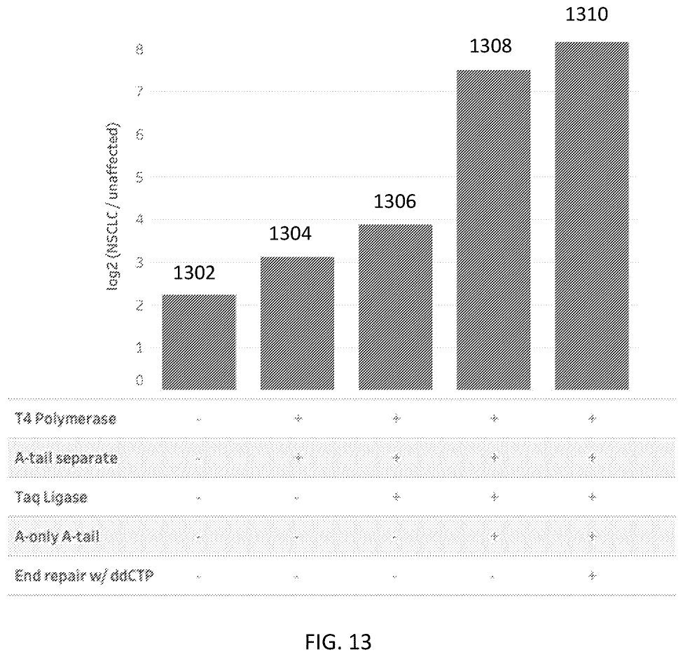

FIG. 13 provides a non-limiting example of the ratio of hypomethylation score for NSCLC samples to that for healthy/unaffected samples (plotted on a log base 2 scale) for the five different library preparation methods summarized in the lower panel of the figure. The data in this plot illustrates the difference in detected methylation status for samples from NSCLC and healthy individuals when the sequence read data was derived from libraries prepared using the indicated combinations of non-strand displacement enzymes, Taq DNA ligase, using only dATP in the tailing reaction, and/or the addition of ddCTP to the end repair and nick/gap repair reactions to minimize the impact of methylation bias. For samples prepared using a conventional sequencing library preparation protocol (1302), the log 2 value for the ratio of hypomethylation score for NSCLC samples to that for healthy/unaffected samples was about 2.25 (i.e., the ratio of hypomethylation scores was about 4.7×). For sequencing libraries prepared using T4 polymerase for end repair and a separate A-tailing reaction as a control (1304), the log 2 value increased to about 3.3. For sequencing libraries prepared using T4 polymerase for end repair, a separate A-tailing reaction, and Taq ligase for nick/gap repair (1306), the log 2 value increased to about 3.9. For sequencing libraries prepared using T4 polymerase for end repair, a separate A-tailing reaction, Taq ligase for nick/gap repair, and where the A-tailing reaction only include dATP (1308), the log 2 value increased to about 7.5. For sequencing libraries prepared using T4 polymerase for end repair, a separate A-tailing reaction, Taq ligase for nick/gap repair, where the A-tailing reaction only include dATP, and where ddCTP was included in the end repair and nick/gap reaction mix (1310), the log 2 value increased to about 8.1 (i.e., the ratio of hypomethylation scores for NSCLC samples to that for healthy/unaffected samples was about 274.4×). The use of the disclosed library preparation steps to mitigate methylation bias during sequencing library preparation eliminated several sources of artificially low levels of methylation detected in the sequence read data for healthy/unaffected samples, and provided greater differentiation in the methylation status detected for affected vs. healthy/unaffected samples. Furthermore, remaining sources of artificially low methylation levels were more localized. Suppression of methylation bias may thus enable more sensitive detection of hypomethylation biomarkers that are indicative of cancer.

### Example 3—Use of 5-Methyl-C in Library Preparation Steps to Mitigate Methylation Bias

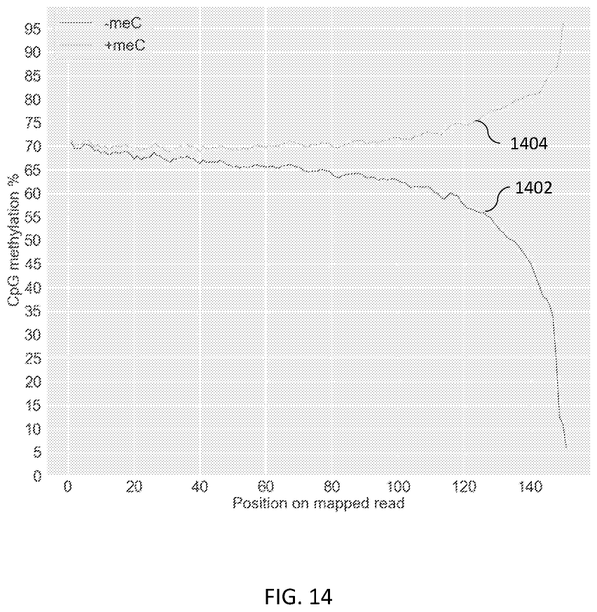

An alternative approach that can be used either alone or in combination with the other approaches to mitigating methylation bias described herein is to use 5-methyl-2′-deoxycytidine-5′-triphosphate (5-methyl-dCTP, or simply 5-methyl-C) to perform end repair during library preparation. In some instances, e.g., when using an enzymatic cytosine conversion reaction for library preparation, other methylated forms of cytosine may also be used (e.g., 5-Hydroxymethyl-2′-deoxycytidine-5′-triphosphate (5-hydroxymethyl-dCTP)). FIG. 14 provides a non-limiting example of percent CpG methylation as a function of sequence read position when DNA sequencing libraries were prepared using non-methylated dCTP (−meC) (curve 1402) or 5-methylated dCTP (+meC) (curve 1404) during end repair. As can be seen, 5-methyl-dCTP can be used to invert the methylation bias signal that is observed when using dCTP. Because the methylated cytosine base would be incorporated at every cytosine base position (not only at CpG sites), one can use the approach to detect which bases have been repaired (e.g., not all cytosine bases would be converted when using bisulfite, etc.). Such an approach may be used, for example, for fragment level correction of methylation bias. In some instances, the use of 5-methyl dCTP may be used instead of ddCTP when performing end repair and/or nick/gap repair.

### Example 4—Quantification of Methylation Bias

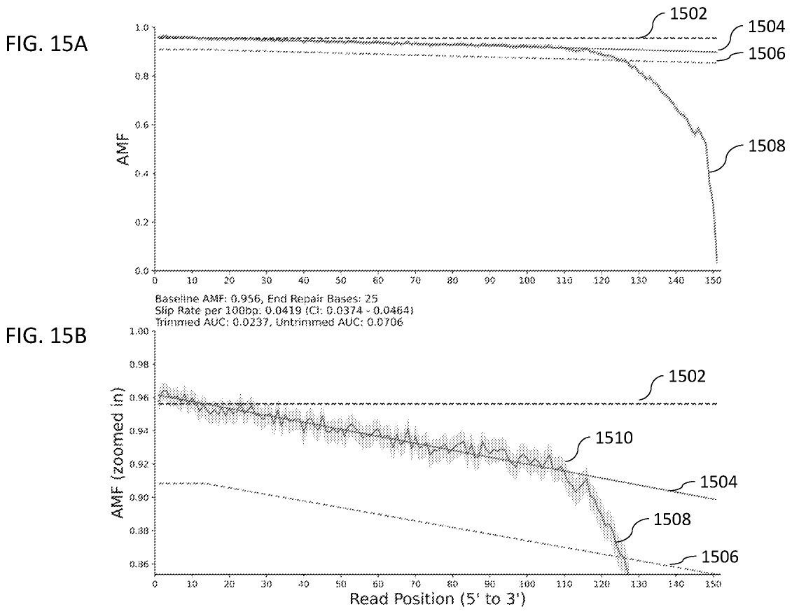

FIGS. 15A-B provide non-limiting examples of data for average methylation fraction (AMF) in healthy/unaffected sample for exemplary methylated control regions (i.e., genomic regions that show high levels of methylation in a wide variety of samples, including both cancer samples and samples from healthy individuals) plotted as a function of position on a sequence read. FIG. 15B presents the same data included in FIG. 15A but plotted on an expanded scale. The curves plotted in FIGS. 15A-B correspond to the baseline level of AMF (dashed line 1502), the “slip rate” (solid line 1504), the end repair threshold (dashed line 1506), the actual AMF data (curve 1508), and the confidence interval (area 1510 centered on solid line 1508). Average methylation fraction values were calculated from the original top (OT) strand aligned sequence reads. Read 2 sequence reads were used to focus on 3′-end related methylation biases. The baseline AMF value (0.956 in this example) was determined as the median AMF value for the first 20 bases at the 5′-end of the sequence read. The “slip rate” (or change in AMF per 100 bases; equal to 0.0419 with a confidence interval of 0.0374 to 0.0464 in this example) was determined based on the slope of the curve opposite the 3′-end of the sequence reads, which may be due to strand displacement, and may also be related to DNA damage. The term “end repair bases” (25 bases in this example) refers to the number of bases for which AMF was consistently below the end repair threshold (dashed line 1306) defined by the “slip rate” (the end repair threshold was defined as 0.95 times the minimum of the slip model (a linear regression of a subset of the bases selected empirically: 40-100 for Read 1, 20-80 for Read 2) and the baseline AMF; the value of 0.95 was chosen semi-empirically to be close to 1 but small enough that variability in typical samples would not be expected to go below the threshold.). Area under the curve (AUC) metrics may be used to quantify the deviation of the observed AMF from the expected baseline AMF value. The “untrimmed” AUC (the total area between the baseline and actual AMF data curves across all sequence read base positions) was 0.0706 in this example. The “trimmed” AUC (the area between the baseline and actual AMF curves across sequence read base positions after excluding data for bases that are computationally trimmed (e.g., a fixed, empirically determined number of bases (5-50 bases) at each end of each read are excluded from methylation calling) from the sequence read data prior to performing methylation calling) was 0.0237 in this example.

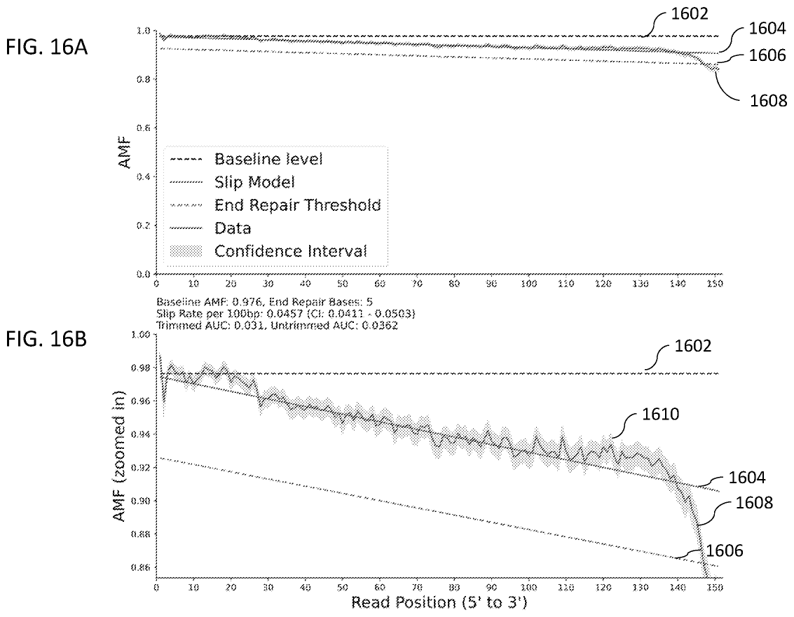

FIGS. 16A-B provide additional non-limiting examples of data for average methylation fraction (AMF) in healthy/unaffected sample for exemplary methylated control regions plotted as a function of position on a sequence read (from Read 1 sequence read data). FIG. 16B presents the same data included in FIG. 16A but plotted on an expanded scale. The curves plotted in FIGS. 16A-B again correspond to the baseline level of AMF (dashed line 1602), the “slip rate” (solid line 1604), the end repair threshold (dashed line 1606), the actual AMF data (curve 1608), and the confidence interval (area 1610 centered on solid line 1608). Average methylation fraction values were calculated from the original top (OT) strand aligned sequence reads from Read 1. The data plotted in FIGS. 16A-B exhibits a complementary pattern to that extracted for the Read 2 sequence reads shown in FIGS. 15A-B. The baseline AMF was 0.976 in this example, slightly higher than that for the data shown in FIGS. 15A-B due to the DNA sequence regions analyzed being further upstream near the 5′-end of the methylated control regions, which are less impacted by slip. The slip rate looks comparable to that for the data shown in FIGS. 15A-B for base positions 40-100. The slip rate was 0.0457 per 100 bases, with a confidence interval of 0.0411 to 0.0503. The number of end repair bases was 5. Fewer bases were impacted by end repair in this case, as the average cfDNA insert size is typically 165-170 bp, which is longer than the length of the read (151 bases here). For Read 2 plots, the 150^{th} base position is close to the 3′ end of the fragment, but in Read 1 plots, the 150^{th} base position is, on average, 15-20 bases away from the 3′ end. The end repair effect is thus weaker in the Read 1 data, and therefore the end repair model does not flag as many bases.

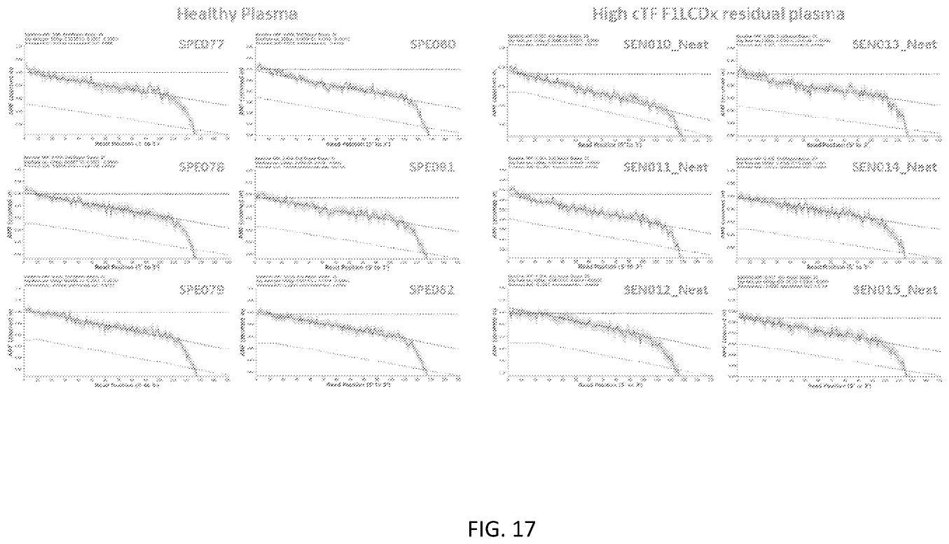

FIG. 17 provides non-limiting examples of methylation bias pattern data that illustrates that methylation bias is consistent in sequence read data derived from many different samples. The two left-most columns include plots of AMF versus sequence read position for sequence read data from plasma samples obtained from healthy individuals. The two right-most columns include plots of AMF versus sequence read position for sequence read data from high (20%+) tumor fraction (high ctDNA) undiluted plasma samples obtained from cancer patients. Each plot provides data for individual plasma samples derived from Read 2, original top strand aligned reads.

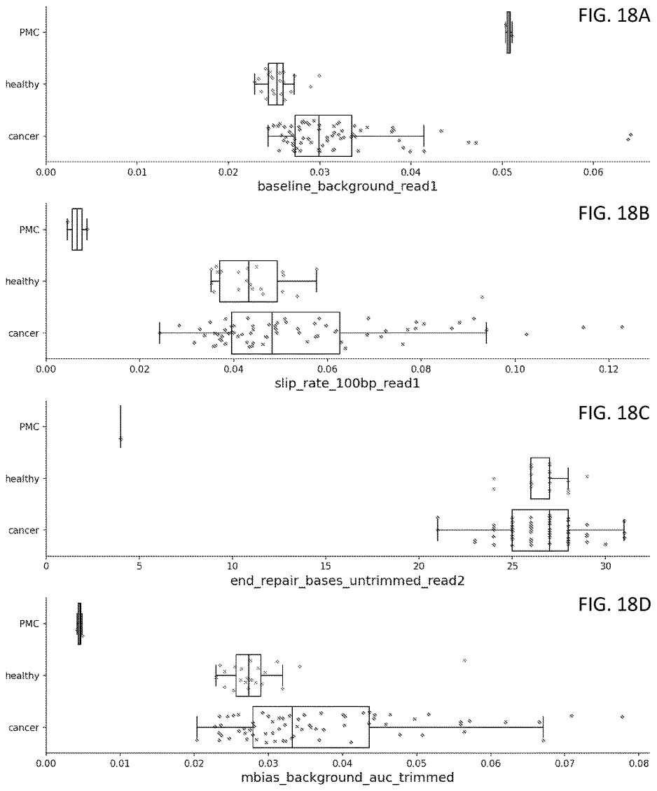

FIGS. 18A-D provide non-limiting examples of plots of baseline AMF background (FIG. 18A), slip rate (FIG. 18B), the number of end repair bases (FIG. 18C), and trimmed AUC (FIG. 18D) observed for sequence read data derived from samples for health individuals and individuals diagnosed with cancer. The data include data for two process matched control (PMC) samples (this DNA was not damaged, so the methylation bias is much smaller), 22 healthy plasma samples, and 70 high-ctDNA plasma samples (mixed cancer types: lung, breast, colorectal, prostate). The sequence read data for samples from cancer patients tended to exhibit more methylation bias, and much more variable methylation bias (perhaps due to the trend towards more DNA damage (e.g., smaller fragment sizes) in high tumor fraction cfDNA samples). The differences in baseline background (i.e., the background=1−AMF baseline) are likely due to genomic region selection issues. For example, PMC samples (negative controls) have known differences with other types of control samples, such as cfDNA samples from healthy subjects. Genomic region sets can be modified to account for these differences to improve the performance of a negative control PMC sample, but this was not done in this case and accounts for the higher baseline AMF in the PMC samples.

### Example 5—Methylation Bias and Base Substitution Error Suppression Using Lowered dCTP Concentrations in End Repair and A-Tailing (ERAT) Reactions

In this example, studies were performed to evaluate the impact of using lower dCTP concentrations for performing end repair and A-tailing (ERAT) reactions on suppression of methylation bias and single base substitution errors. As illustrated in FIG. 8D above, the inclusion of ddCTP in ERAT reactions excludes some molecules from sequencing. It was hypothesized that the use of less stringent artifact suppression may boost sequencing library complexity.

Six different nucleotide mixtures comprising different dDTP:dCTP ratios (where dDTP represents the three deoxynucleotide triphosphates other than dCTP) were used for performing ERAT reactions, and their impact on suppression of methylation bias and single base substitution errors was evaluated. The six nucleotide mixtures included mixtures comprising ratios of: (i) dDTP:dCTP=1:1 (“dC_par”), (ii) dDTP:dCTP=10:1, (iii) dDTP:dCTP=50:1, (iv) dDTP:dCTP=100:1, (v) dDTP:dCTP=500:1, and (vi) dDTP+ddCTP:dCTP=50:1 (“ddC_50”; i.e., the dDTPs (A, T, and G) and ddCTP were all at the same concentration (1:1), while dCTP was included at 1/50th of that concentration). The evaluation was performed on three different nucleic acid sample extracts, with three technical replicates performed per reaction condition. The quality of the input nucleic acid sample extracts was evaluated and confirmed by electrophoretic separation using an Agilent Technologies (Santa Clara, CA) TapeStation system.

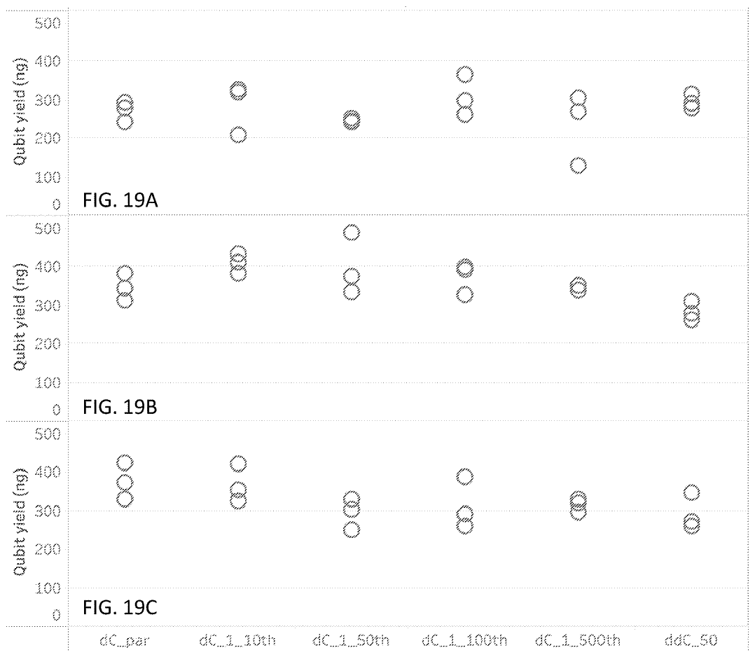

Sequencing library preparation was performed using the workflow illustrated in FIG. 1B. Use of restrictive nucleotide conditions for performing ERAT reactions have previously demonstrated reductions in library yield (see, e.g., FIG. 8B above). FIGS. 19A-C provide non-limiting examples of data for nucleic acid yield for three different sample extracts (FIG. 19A: extract identification number OT04788; FIG. 19B: extract identification number OT04789; FIG. 19C: extract identification number OT04796) plotted as a function of the lowered dCTP conditions used to perform end repair and A tailing during sequencing library preparation. As can be seen, the sequencing library yields were fairly uniform across the range of dDTP:dCTP ratios evaluated.

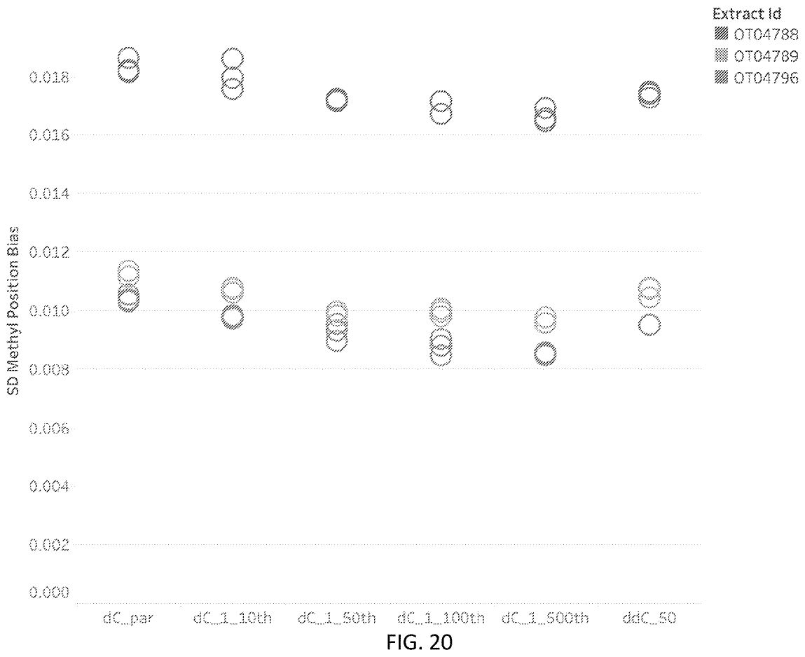

As noted above with respect to FIG. 11, varying the choice of polymerase, the choice of ligase, the mixture of nucleotides used to perform the tailing reaction, and the ddCTP concentration used to perform end repair, tailing, and adapter ligation had an additive effect on the suppression of the observed SD methylation position bias. As illustrated by the data presented in FIGS. 10A-B, the methylation bias profile moves visibly in accordance with the SD methylation position bias metric. FIG. 20 provides a non-limiting example of data for SD methylation position bias for the three different sample extracts plotted as a function of the conditions used to perform end repair and A tailing during sequencing library preparation. As can be seen in this figure, significant reductions in methylation bias were observed for all six of the nucleotide mixtures used for performing ERAT reactions (compare to the control data in FIG. 11), with an apparent minimum value of observed methylation bias (i.e., a maximum value of methylation bias suppression) in the dDTP:dCTP range of about 50:1 to 500:1.

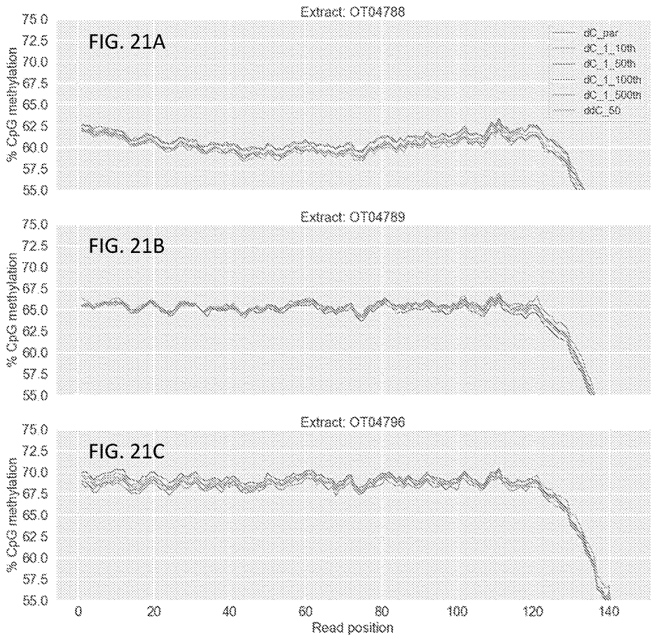

FIGS. 21A-C provide non-limiting examples of data for percent CpG methylation for the three different sample extracts plotted as a function of sequence read position for the different conditions used to perform end repair and A tailing during sequencing library preparation. As can be seen, despite some systematic difference between the samples, all sample extracts exhibited a similar methylation bias profile for all nucleotide mixtures used to perform ERAT reactions.

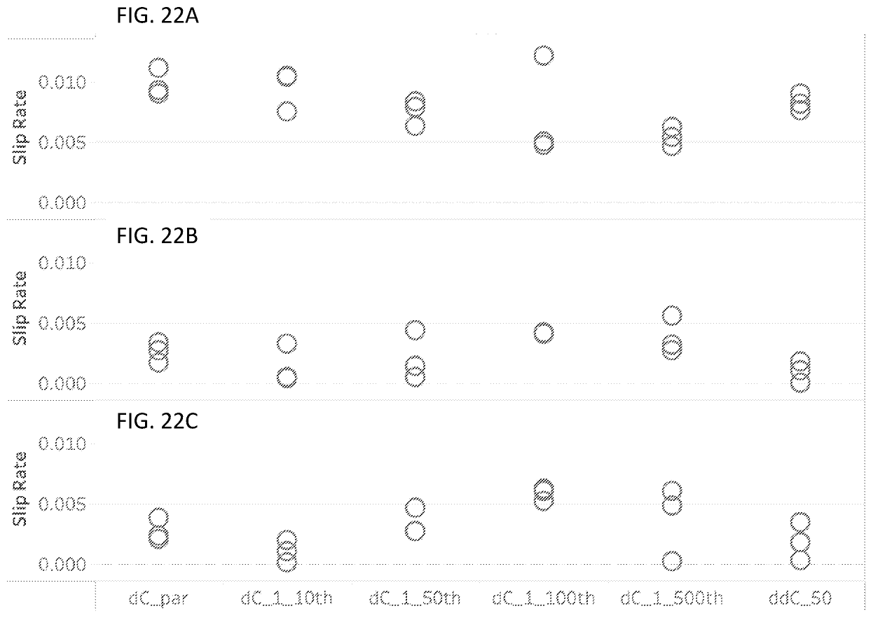

FIGS. 22A-C provide non-limiting examples of data for slip rate (determined as described elsewhere herein) for the three different sample extracts (FIG. 22A: extract identification number OT04788; FIG. 22B: extract identification number OT04789; FIG. 22C: extract identification number OT04796) plotted as a function of the conditions used to perform end repair and A tailing during sequencing library preparation. For sequence read 1 data, nucleotide positions 40-100 were used to determine the slip rate (i.e., the slope of the curve as determined using a regression model). For sequence read 2 data, nucleotide positions 20-80 were used to determine the slip rate. As can be seen, the slip rate was fairly uniform for the different nucleotide mixtures used to perform ERAT reactions.

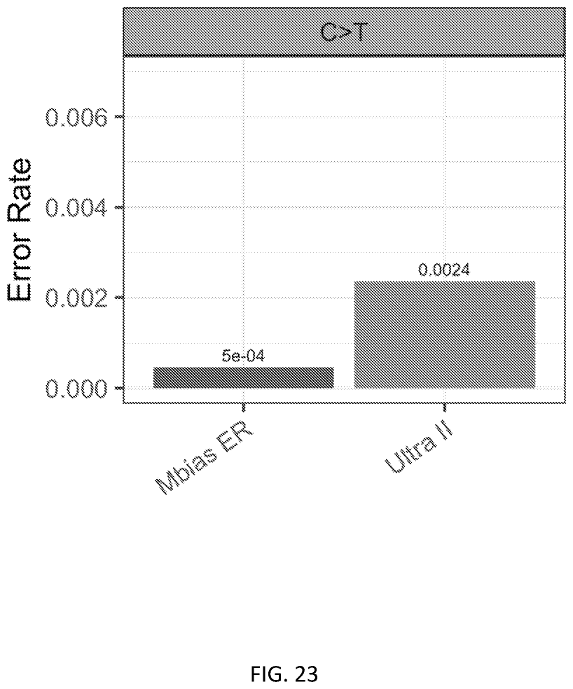

FIG. 23 provides a non-limiting example of data for single base C-to-T substitution error rate plotted as a function of the conditions used to perform end repair and A tailing during sequencing library preparation of cell line genomic DNA. As can be seen, use of ddCTP in the nucleotide mixture used to perform end repair resulted in a substantial decrease in single base C-to-T substitution error rate as compared to that observed when end repair was performed using the Ultra™ II End Repair/dA-Tailing protocol from New England Biolabs (Ipswich, MA).

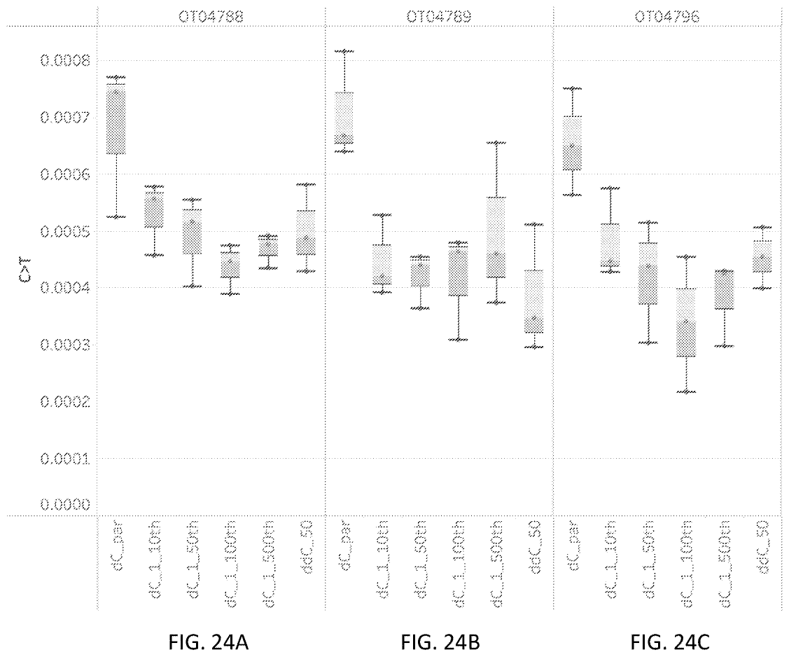

FIGS. 24A-C provide a non-limiting example of data for erroneous error rates for C→T single base substitutions plotted for the three different sample extracts (FIG. 24A: extract identification number OT04788; FIG. 24B: extract identification number OT04789; FIG. 24C: extract identification number OT04796) as a function of the conditions used to perform end repair and A tailing during sequencing library preparation. As can be seen, significantly lower single base substitution error rates were observed (compare to the Ultra™ II End Repair/dA-Tailing protocol control data shown in FIG. 23) for all six different nucleotide mixtures used to perform ERAT reactions.

Collectively, this data indicates that significant improvements in suppression of methylation bias and base substitution error can be achieved using lowered dCTP concentrations to perform end repair and A-tailing (ERAT) reactions.

### Exemplary Implementations

Exemplary implementations of the methods described herein include:
1. A method comprising:
   extracting one or more nucleic acid fragments from a sample obtained from a subject;
   performing at least one of:
      a first end repair reaction to repair fragmentation damage to one or more nucleic acid fragment ends, wherein the first end repair reaction comprises the use of a non-strand-displacing polymerase; or
      a second end repair reaction to repair fragmentation damage to one or more nucleic acid fragment ends, wherein the second end repair reaction comprises the use of a chain termination mechanism;
   performing at least one of:
      a tailing reaction to add an overhanging polynucleotide strand to the one or more end-repaired nucleic acid fragments, wherein the tailing reaction comprises the use of a single deoxynucleotide triphosphate (dNTP); or
      a nick/gap repair reaction to fill in single-stranded nicks or gaps in the one or more end-repaired nucleic acid fragments, wherein the nick/gap repair reaction comprises the use of a ligase
   to generate a plurality of modified nucleic acid fragments; and
   performing methylation analysis of the plurality of modified nucleic acid fragments.
2. The method of clause 1, wherein the methylation analysis comprises performing a cytosine conversion reaction on the plurality of modified nucleic acid fragments to generate a plurality of converted nucleic acid fragments.
3. The method of clause 2, wherein the methylation analysis comprises sequencing the plurality of converted nucleic acid fragments to generate a plurality of sequence reads.
4. The method of clause 3, further comprising performing a sequence read analysis based on the plurality of sequence reads to determine a methylation signature of the subject.
5. The method of any one of clauses 1 to 4, comprising performing both a first end repair reaction and a second end repair reaction.
6. The method of any one of clauses 1 to 5, comprising performing both a tailing reaction comprising the use of a single dNTP and a nick/gap repair reaction comprising the use of a ligase.
7. The method of any one of clauses 1 to 6, wherein the methylation analysis comprises a restriction enzyme-based, affinity enrichment-based, bisulfite conversion-based, or enzymatic conversion-based methylation sequencing method.
8. The method of any one of clauses 1 to 7, further comprising ligating one or more adapters to the plurality of modified nucleic acid fragments.
9. The method of clause 8, wherein the one or more adapters comprise one or more adapters comprising an overhanging poly-T sequence.
10. The method of clause 8, wherein the one or more adapters comprise one or more sequencing adapters.
11. The method of clause 10, wherein the one or more sequencing adapters comprise one or more methylated stubby adapters, flow cell adapters, read 1 sequencing adapters, read 2 sequencing adapters, or any combination thereof.
12. The method of clause 8, wherein the one or more adapters comprise one or more barcodes.
13. The method of any one of clauses 1 to 11, further comprising ligating one or more barcodes to the plurality of modified nucleic acid fragments.
14. The method of clause 12 or clause 13, wherein the one or more barcodes comprise a library index, a sample barcode, a cell barcode, a target-specific barcode, a DNA fragment barcode, a unique molecular index, or any combination thereof.
15. The method of any one of clauses 2 to 14, wherein the cytosine conversion reaction is used to convert non-methylated cytosine to uracil.
16. The method of any one of clauses 2 to 15, wherein the cytosine conversion reaction comprises a chemical conversion reaction.
17. The method of clause 16, wherein the chemical conversion reaction comprises a bisulfite conversion reaction.
18. The method of any one of clauses 2 to 15, wherein the cytosine conversion reaction comprises an enzymatic conversion reaction.
19. The method of clause 18, wherein the enzymatic conversion reaction comprises the use of a tet methylcytosine dioxygenase 2 (TET2) enzyme to oxidize 5-methyl-cytosine (5mC) or 5-hydroxymethyl-cytosine (5hmC) to 5-carboxycytosine (5caC).
20. The method of clause 18 or clause 19, wherein the enzymatic conversion reaction further comprises the use of a combination of TET2 and T4 β-glucosyltransferase (T4-βGT) enzymes to convert 5-methyl-cytosine (5mC) or 5-hydroxymethyl-cytosine (5hmC) to 5-(D-glucosyl)oxymethyl-cytosine.
21. The method of any one of clauses 18 to 20, wherein the enzymatic conversion reaction comprises the use of an Apolipoprotein B mRNA Editing Catalytic Polypeptide-like (APOBEC) enzyme.
22. The method of any one of clauses 1 to 16, further comprising performing a nucleic acid amplification reaction to generate amplified nucleic acid molecules that are derived from the plurality of modified nucleic acid fragments.
23. The method of clause 22, wherein the nucleic acid amplification reaction comprises a polymerase chain reaction (PCR).
24. The method of clause 22, wherein the nucleic acid amplification reaction comprises a rolling circle amplification (RCA) reaction.
25. The method of any one of clauses 22 to 24, further comprising capturing a subset of nucleic acid molecules from the amplified nucleic acid molecules and performing methylation analysis on the subset.
26. The method of any one of clauses 1 to 25, wherein the one or more nucleic acid fragments comprises double-stranded nucleic acid fragments.
27. The method of any one of clauses 1 to 26, wherein the non-strand-displacing polymerase comprises a non-strand-displacing DNA polymerase.
28. The method of clause 27, wherein the non-strand displacing DNA polymerase comprises T4 DNA polymerase.
29. The method of any one of clauses 1 to 28, wherein the chain termination mechanism comprises the use of a chain termination nucleotide in the end repair reaction.
30. The method of clause 29, wherein the chain termination nucleotide comprises a 2′,3′-dideoxyribonucleoside 5′-triphosphate (ddNTP).
31. The method of clause 30, wherein the ddNTP comprises a 2′,3′-dideoxycytidine 5′-triphosphate (ddCTP), 2′,3′-dideoxyguanosine 5′-triphosphate (ddGTP), 2′,3′-dideoxythymidine 5′-triphosphate (ddTTP), 2′,3′-dideoxyadenosine 5′-triphosphate (ddATP), or any combination thereof.
32. The method of clause 30 or clause 31, wherein a ratio of ddNTP concentration to a corresponding dNTP concentration used to perform the end repair reaction is less than 20×, 30×, 40×, 50×, or 60×, 70×, 80×, 100×, 120×, or 140×.
33. The method of any one of clauses 1 to 32, wherein the chain termination mechanism comprises omitting a dNTP from the end repair reaction.
34. The method of any one of clauses 1 to 32, wherein the chain termination mechanism comprises including only a limiting amount of a dNTP in the end repair reaction.
35. The method of any one of clauses 1 to 34, wherein one or more modified nucleotides are used as part of performing the end repair reaction to indicate nucleic acid sequence regions where end repair has occurred.
36. The method of any one of clauses 1 to 35, wherein one or more modified nucleotides are used as part of performing the tailing reaction to identify the overhanging poly-nucleotide strand added to the modified nucleic acid fragments.
37. The method of any one of clauses 1 to 36, wherein one or more modified nucleotides are used as part of performing the nick/gap repair reaction to identify filled in portions of the modified nucleic acid fragments.
38. The method of any one of clauses 35 to 37, wherein the one or more modified nucleotides comprise 5-methyldeoxycytidine 5′-triphosphate (5-methyl dCTP), 5-hydroxymethyldeoxycytidine 5′-triphosphate, deoxyuradine 5′-triphosphosphate, oxoguanosine 5′-triphosphate, or any combination thereof.
39. The method of any one of clauses 1 to 38, wherein the tailing reaction comprises the use of 2′-deoxyadenosine 5′-triphosphate (dATP).
40. The method of any one of clauses 1 to 38, wherein the tailing reaction comprises the use of 2′-deoxythymidine 5′-triphosphate (dTTP).
41. The method of any one of clauses 1 to 38, wherein the tailing reaction comprises the use of 2′-deoxycytidine 5′-triphosphate (dCTP).
42. The method of any one of clauses 1 to 38, wherein the tailing reaction comprises the use of 2′-deoxyguanosine 5′-triphosphate (dGTP).
43. The method of any one of clauses 1 to 42, wherein the tailing reaction comprises the use of T4 DNA polymerase, T4 Polynucleotide Kinase Reaction (T4 PNK), Taq polymerase, Klenow Fragment (3′→5′ exo-), Sulfolobus DNA polymerase IV, or any combination thereof.
44. The method of any one of clauses 1 to 43, wherein the tailing reaction is omitted and a downstream blunt ligation step is used to ligate one or more adapters to the one or more nucleic acid fragments.
45. The method of any one of clauses 1 to 44, wherein the ligase comprises a DNA ligase.
46. The method of clause 45, wherein the DNA ligase comprises Taq DNA ligase, T4 DNA ligase, 9°NÔ DNA ligase, T3 DNA ligase, or any combination thereof.
47. The method of any one of clauses 1 to 46, wherein sequence read data obtained by sequencing nucleic acid molecules derived from the plurality of modified nucleic acid fragments exhibits reduced methylation bias compared to that obtained by sequencing a conventionally-prepared DNA sequencing library.
48. The method of clause 47, wherein the reduction in methylation bias is greater than 5%, 10%, 15%, 20%, 25%, 30%, or 35% as measured by standard deviation (SD) methyl position bias.
49. The method of any one of clauses 1 to 48, wherein the methylation analysis is performed using a next-generation sequencer.
50. The method of clause 49, wherein the methylation analysis further comprises sequencing, using the next-generation sequencer, the plurality of modified nucleic acid fragments.
51. The method of clause 49 or clause 50, wherein the methylation analysis comprises using the next-generation sequencing to perform whole genome sequencing, whole exome sequencing, or targeted sequencing.
52. The method of any one of clauses 3 to 51, further comprising determining a methylation status for each of one or more genomic loci based on sequence read data for the plurality of sequence reads.
53. The method of any one of clauses 3 to 52, further comprising screening, detecting, diagnosing, confirming a diagnosis of, or monitoring disease in the subject based on sequence read data for the plurality of sequence reads.
54. The method of clause 53, wherein the screening, detecting, diagnosing, confirming a diagnosis, or monitoring of disease is performed with improved accuracy due to a reduction in methylation bias in the sequence read data compared to that obtained by sequencing a conventionally-prepared DNA sequencing library.
55. The method of any one of clauses 3 to 54, further comprising detecting minimum residual disease in the subject based on sequence read data for the plurality of sequence reads.
56. The method of any one of clauses 53 to 55, wherein the disease is cancer.
57. The method of any one of clauses 1 to 56, wherein the methylation analysis is used to detect hypomethylated or hypermethylated genomic regions in cancer patients.
58. The method of any one of clauses 1 to 57, wherein the one or more nucleic acid fragments comprise one or more DNA fragments.
59. A method comprising:
   extracting a plurality of DNA fragments from a sample obtained from a subject;
   performing at least one of:
      a first end repair reaction to repair fragmentation damage to one or more DNA fragment ends, wherein the first end repair reaction comprises the use of a non-strand-displacing DNA polymerase; or
      a second end repair reaction to repair fragmentation damage to one or more DNA fragment ends, wherein the second end repair reaction comprises the use of a chain termination mechanism;
   performing at least one of:
      a tailing reaction to add an overhanging polynucleotide strand to the one or more end-repaired DNA fragments, wherein the tailing reaction comprises the use of a single dNTP; or
      a nick/gap repair reaction to fill in single-stranded nicks or gaps in the one or more end-repaired DNA fragments, wherein the nick/gap repair reaction comprises the use of a DNA ligase
   to generate a plurality of modified DNA fragments;
   ligating one or more adapters onto one or more modified DNA fragments from the plurality of modified DNA fragments;
   performing a cytosine conversion reaction on the one or more ligated DNA fragments to generate one or more converted DNA fragments;
   amplifying the one or more converted DNA fragments;
   capturing one or more amplified converted DNA fragments;
   sequencing, by a sequencer, the one or more captured converted DNA fragments to obtain a plurality of sequence reads that represent the one or more captured converted DNA fragments;
   receiving, at one or more processors, sequence read data for the plurality of sequence reads; and
   performing a methylation analysis of nucleic acid molecules derived from the plurality of modified DNA fragments based on the plurality of sequence reads.
60. The method of clause 59, wherein the methylation analysis comprises determining a methylation signature for the subject.
61. The method of clause 59 or clause 60, comprising performing both a first end repair reaction and a second end repair reaction.
62. The method of any one of clauses 59 to 61, comprising performing both a tailing reaction comprising the use of a single dNTP and a nick/gap repair reaction comprising the use of a DNA ligase.
63. The method of any one of clauses 59 to 62, wherein the subject is suspected of having or is determined to have cancer.
64. The method of clause 63, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
65. The method of clause 63, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
66. The method of any one of clauses 63 to 65, further comprising treating the subject with an anti-cancer therapy.
67. The method of clause 66, wherein the anti-cancer therapy comprises a targeted anti-cancer therapy.
68. The method of clause 67, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
69. The method of any one of clauses 59 to 68, further comprising obtaining the sample from the subject.
70. The method of any one of clauses 59 to 69, wherein the sample comprises a tissue biopsy sample, a liquid biopsy sample, or a normal control.
71. The method of clause 70, wherein the sample is a liquid biopsy sample and comprises blood, plasma, cerebrospinal fluid, sputum, stool, urine, or saliva.
72. The method of clause 70, wherein the sample is a liquid biopsy sample and comprises circulating tumor cells (CTCs).
73. The method of clause 70, wherein the sample is a liquid biopsy sample and comprises cell-free DNA (cfDNA), circulating tumor DNA (ctDNA), or any combination thereof.
74. The method of any one of clauses 59 to 73, wherein the plurality of DNA fragments comprises a mixture of tumor nucleic acid molecules and non-tumor nucleic acid molecules.
75. The method of clause 74, wherein the tumor nucleic acid molecules are derived from a tumor portion of a heterogeneous tissue biopsy sample, and the non-tumor nucleic acid molecules are derived from a normal portion of the heterogeneous tissue biopsy sample.
76. The method of clause 74, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
77. The method of any one of clauses 59 to 76, wherein the one or more adapters comprise amplification primers, sequencing adapter sequences, flow cell adaptor sequences, substrate adapter sequences, or sample index sequences.
78. The method of any one of clauses 59 to 77, wherein the captured DNA fragments are captured from the amplified DNA fragments by hybridization to one or more bait molecules.
79. The method of clause 78, wherein the one or more bait molecules comprise one or more nucleic acid molecules, each comprising a region that is complementary to a region of a captured DNA fragment.
80. The method of any one of clauses 59 to 79, wherein amplifying converted DNA fragments comprises performing a polymerase chain reaction (PCR) amplification technique, a non-PCR amplification technique, or an isothermal amplification technique.
81. The method of any one of clauses 59 to 80, wherein the sequencing comprises use of a massively parallel sequencing (MPS) technique, whole genome sequencing (WGS), whole exome sequencing, targeted sequencing, direct sequencing, or Sanger sequencing technique.
82. The method of clause 81, wherein the sequencing comprises massively parallel sequencing, and the massively parallel sequencing technique comprises next generation sequencing (NGS).
83. The method of any one of clauses 59 to 82, wherein the sequencer comprises a next generation sequencer.
84. The method of any one of clauses 59 to 83, wherein one or more of the plurality of sequencing reads overlap one or more gene loci within one or more subgenomic intervals in the sample.
85. The method of clause 84, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
86. The method of clause 84 or clause 85, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
87. The method of clause 84 or clause 85, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.
88. The method of any one of clauses 59 to 87, further comprising generating, by the one or more processors, a report indicating a result of the methylation analysis.
89. The method of clause 88, further comprising transmitting the report to a healthcare provider.
90. The method of clause 89, wherein the report is transmitted via a computer network or a peer-to-peer connection.
91. A method for diagnosing a disease, the method comprising:
   diagnosing that a subject has the disease based on a methylation analysis or a determination of a methylation signature of a sample from the subject, wherein the methylation analysis or the methylation signature is determined according to the method of any one of clauses 1 to 90.
92. A method of selecting an anti-cancer therapy, the method comprising:
   responsive to a methylation analysis or a determination of a methylation signature of a sample from a subject, selecting an anti-cancer therapy for the subject, wherein the methylation analysis or the methylation signature is determined according to the method of any one of clauses 1 to 90.
93. A method of treating a cancer in a subject, comprising:
   responsive to a methylation analysis or a determination of a methylation signature of a sample from the subject, administering an effective amount of an anti-cancer therapy to the subject, wherein the methylation analysis or the methylation signature is determined according to the method of any one of clauses 1 to 90.
94. A method for monitoring cancer progression or recurrence in a subject, the method comprising:
   performing a first methylation analysis or determining a first methylation signature for a first sample obtained from the subject at a first time point according to the method of any one of clauses 1 to 90;
   performing a second methylation analysis or determining a second methylation signature for a second sample obtained from the subject at a second time point; and comparing the first methylation analysis or methylation signature to the second methylation analysis or methylation signature, thereby monitoring the cancer progression or recurrence.
95. The method of clause 94, wherein the second methylation analysis or methylation signature for the second sample is determined according to the method of any one of clauses 1 to 90.
96. The method of clause 94 or clause 95, further comprising selecting an anti-cancer therapy for the subject in response to the cancer progression.
97. The method of clause 94 or clause 95, further comprising administering an anti-cancer therapy to the subject in response to the cancer progression.
98. The method of clause 94 or clause 95, further comprising adjusting an anti-cancer therapy for the subject in response to the cancer progression.
99. The method of any one of clauses 96 to 98, further comprising adjusting a dosage of the anti-cancer therapy or selecting a different anti-cancer therapy in response to the cancer progression.
100. The method of clause 99, further comprising administering the adjusted anti-cancer therapy to the subject.
101. The method of any one of clauses 94 to 100, wherein the first time point is before the subject has been administered an anti-cancer therapy, and wherein the second time point is after the subject has been administered the anti-cancer therapy.
102. The method of any one of clauses 94 to 101, wherein the subject has a cancer, is at risk of having a cancer, is being routine tested for cancer, or is suspected of having a cancer.
103. The method of any one of clauses 94 to 102, wherein the cancer is a solid tumor.
104. The method of any one of clauses 94 to 102, wherein the cancer is a hematological cancer.
105. The method of any one of clauses 96 to 104, wherein the anti-cancer therapy comprises chemotherapy, radiation therapy, immunotherapy, a targeted therapy, or surgery.
106. The method of any one of clauses 1 to 90, further comprising determining, identifying, or applying a methylation analysis result or methylation signature for the sample as a diagnostic value associated with the sample.
107. The method of any one of clauses 1 to 90, further comprising generating a genomic profile for the subject based on the determination of a methylation analysis result or methylation signature.
108. The method of clause 107, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
109. The method of clause 107 or clause 108, wherein the genomic profile for the subject further comprises results from a nucleic acid sequencing-based test.
110. The method of any one of clauses 107 to 109, further comprising selecting an anti-cancer therapy, administering an anti-cancer therapy, or applying an anti-cancer therapy to the subject based on the generated genomic profile.
111. The method of any one of clauses 1 to 90, wherein the determination of a methylation analysis result or a methylation signature for the sample is used in making suggested treatment decisions for the subject.
112. The method of any one of clauses 1 to 90, wherein the determination of a methylation analysis result or a methylation signature for the sample is used in applying or administering a treatment to the subject.
113. A method comprising:
   extracting a plurality of nucleic acid molecules from a sample obtained from a subject;
   performing at least one of:
      a first end repair reaction to repair fragmentation damage to one or more ends of the nucleic acid molecules, wherein the first end repair reaction comprises the use of a non-strand-displacing DNA polymerase; or
      a second end repair reaction to repair fragmentation damage to the one or more ends, wherein the second end repair reaction comprises the use of a chain termination mechanism;
   performing at least one of:
      a tailing reaction to add an overhanging polynucleotide strand to the one or more end-repaired nucleic acid molecules, wherein the tailing reaction comprises the use of a single deoxynucleotide triphosphate (dNTP); or
      a nick/gap repair reaction to fill in single-stranded nicks or gaps in the one or more end-repaired nucleic acid molecules, wherein the nick/gap repair reaction comprises the use of a DNA ligase
   to generate a plurality of modified nucleic acid molecules; and
   performing sequence analysis of the modified nucleic acid molecules.
114. A system comprising:
   an extractor configured to extract a plurality of a plurality of nucleic acid molecules from a sample obtained from a subject;
   an automated library preparation device configured to execute one or more processes that when executed perform one or more of:
      a first end repair reaction to repair fragmentation damage to one or more ends of the nucleic acid molecules, wherein the first end repair reaction comprises the use of a non-strand-displacing DNA polymerase; or
      a second end repair reaction to repair fragmentation damage to the one or more ends, wherein the second end repair reaction comprises the use of a chain termination mechanism;
   performing at least one of:
      a tailing reaction to add an overhanging polynucleotide strand to the one or more end-repaired nucleic acid molecules, wherein the tailing reaction comprises the use of a single deoxynucleotide triphosphate (dNTP); or
      a nick/gap repair reaction to fill in single-stranded nicks or gaps in the one or more end-repaired nucleic acid molecules, wherein the nick/gap repair reaction comprises the use of a DNA ligase
   to generate a plurality of modified nucleic acid molecules;
   a sequencer configured to sequence the modified nucleic acid molecules and generate one or more sequence modified nucleic acid sequence reads representing the nucleic acid sequence of the modified nucleic acid molecule; and
   a computational analysis platform including one or more processors including a memory that stores one or more processes, the processes when executed configured to analyze the modified nucleic acid reads and identify one or more alterations or epigenetic signatures in the modified nucleic acid molecules.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method comprising:
extracting one or more nucleic acid fragments from a sample obtained from a subject;
performing at least one of:
a first end repair reaction to repair fragmentation damage to one or more nucleic acid fragment ends, wherein the first end repair reaction comprises the use of a non-strand-displacing polymerase; or
a second end repair reaction to repair fragmentation damage to one or more nucleic acid fragment ends, wherein the second end repair reaction comprises the use of a chain termination mechanism;
performing at least one of:
a tailing reaction to add an overhanging polynucleotide strand to the one or more end-repaired nucleic acid fragments, wherein the tailing reaction comprises the use of a single deoxynucleotide triphosphate (dNTP); or
a nick/gap repair reaction to fill in single-stranded nicks or gaps in the one or more end-repaired nucleic acid fragments, wherein the nick/gap repair reaction comprises the use of a ligase
to generate a plurality of modified nucleic acid fragments; and
performing methylation analysis of the plurality of modified nucleic acid fragments.

2. The method of claim 1, wherein the methylation analysis comprises performing a cytosine conversion reaction on the plurality of modified nucleic acid fragments to generate a plurality of converted nucleic acid fragments.

3. The method of claim 2, wherein the methylation analysis comprises sequencing the plurality of converted nucleic acid fragments to generate a plurality of sequence reads.

4. The method of claim 3, further comprising performing a sequence read analysis based on the plurality of sequence reads to determine a methylation signature of the subject.

5. The method of claim 1, comprising performing both a first end repair reaction and a second end repair reaction.

6. The method of claim 1, comprising performing both a tailing reaction comprising the use of a single dNTP and a nick/gap repair reaction comprising the use of a ligase.

7. The method of claim 1, wherein the methylation analysis comprises a restriction enzyme-based, affinity enrichment-based, bisulfite conversion-based, or enzymatic conversion-based methylation sequencing method.

8. The method of claim 1, further comprising ligating one or more adapters to the plurality of modified nucleic acid fragments.

9. The method of claim 8, wherein the one or more adapters comprise one or more barcodes.

10. The method of claim 9, wherein the one or more barcodes comprise a library index, a sample barcode, a cell barcode, a target-specific barcode, a DNA fragment barcode, a unique molecular index, or any combination thereof.

11. The method of claim 2, wherein the cytosine conversion reaction comprises a chemical conversion reaction or an enzymatic conversion reaction.

12. The method of claim 1, wherein the one or more nucleic acid fragments comprises double-stranded nucleic acid fragments.

13. The method of claim 1, wherein the non-strand-displacing polymerase comprises a non-strand-displacing DNA polymerase.

14. The method of claim 1, wherein the chain termination mechanism comprises the use of a chain termination nucleotide in the end repair reaction.

15. The method of claim 1, wherein the chain termination mechanism comprises omitting a dNTP from the end repair reaction.

16. The method of claim 1, wherein one or more modified nucleotides are used as part of performing the end repair reaction to indicate nucleic acid sequence regions where end repair has occurred.

17. The method of claim 1, wherein one or more modified nucleotides are used as part of performing the tailing reaction to identify the overhanging poly-nucleotide strand added to the modified nucleic acid fragments.

18. The method of claim 1, wherein one or more modified nucleotides are used as part of performing the nick/gap repair reaction to identify filled in portions of the modified nucleic acid fragments.

19. The method of claim 1, wherein the tailing reaction comprises the use of T4 DNA polymerase, T4 Polynucleotide Kinase Reaction (T4 PNK), Taq polymerase, Klenow Fragment (3′→5′ exo-), Sulfolobus DNA polymerase IV, or any combination thereof.

20. The method of claim 1, wherein the tailing reaction is omitted and a downstream blunt ligation step is used to ligate one or more adapters to the one or more nucleic acid fragments.

21. The method of claim 1, wherein the ligase comprises a DNA ligase, and wherein the DNA ligase comprises Taq DNA ligase, T4 DNA ligase, 9oN□ DNA ligase, T3 DNA ligase, or any combination thereof.

22. The method of claim 1, wherein sequence read data obtained by sequencing nucleic acid molecules derived from the plurality of modified nucleic acid fragments exhibits reduced methylation bias compared to that obtained by sequencing a conventionally-prepared DNA sequencing library.

23. The method of claim 22, wherein the reduction in methylation bias is greater than 5%, 10%, 15%, 20%, 25%, 30%, or 35% as measured by standard deviation (SD) methyl position bias.

24. The method of claim 3, further comprising determining a methylation status for each of one or more genomic loci based on sequence read data for the plurality of sequence reads.

25. The method of claim 3, further comprising screening, detecting, diagnosing, confirming a diagnosis of, or monitoring disease in the subject based on sequence read data for the plurality of sequence reads.

26. The method of claim 25, wherein the screening, detecting, diagnosing, confirming a diagnosis, or monitoring of disease is performed with improved accuracy due to a reduction in methylation bias in the sequence read data compared to that obtained by sequencing a conventionally-prepared DNA sequencing library.

27. The method of claim 3, further comprising detecting minimum residual disease in the subject based on sequence read data for the plurality of sequence reads.

28. The method of claim 25, wherein the disease is cancer.

29. The method of claim 1, wherein the methylation analysis is used to detect hypomethylated or hypermethylated genomic regions in cancer patients.

30. A method comprising:
extracting a plurality of DNA fragments from a sample obtained from a subject;
performing at least one of:
a first end repair reaction to repair fragmentation damage to one or more DNA fragment ends, wherein the first end repair reaction comprises the use of a non-strand-displacing DNA polymerase; or
a second end repair reaction to repair fragmentation damage to one or more DNA fragment ends, wherein the second end repair reaction comprises the use of a chain termination mechanism;
performing at least one of:
a tailing reaction to add an overhanging polynucleotide strand to the one or more end-repaired DNA fragments, wherein the tailing reaction comprises the use of a single dNTP; or
a nick/gap repair reaction to fill in single-stranded nicks or gaps in the one or more end-repaired DNA fragments, wherein the nick/gap repair reaction comprises the use of a DNA ligase
to generate a plurality of modified DNA fragments;
ligating one or more adapters onto one or more modified DNA fragments from the plurality of modified DNA fragments;
performing a cytosine conversion reaction on the one or more ligated DNA fragments to generate one or more converted DNA fragments;
amplifying the one or more converted DNA fragments;
capturing one or more amplified converted DNA fragments;
sequencing, by a sequencer, the one or more captured converted DNA fragments to obtain a plurality of sequence reads that represent the one or more captured converted DNA fragments;
receiving, at one or more processors, sequence read data for the plurality of sequence reads; and
performing a methylation analysis of nucleic acid molecules derived from the plurality of modified DNA fragments based on the plurality of sequence reads.

31. The method of claim 30, wherein the methylation analysis comprises determining a methylation signature for the subject.

32. The method of claim 31, wherein the determination of the methylation signature is used in making suggested treatment decisions for the subject.

33. A method comprising:
extracting a plurality of nucleic acid molecules from a sample obtained from a subject;
performing at least one of:
a first end repair reaction to repair fragmentation damage to one or more ends of the nucleic acid molecules, wherein the first end repair reaction comprises the use of a non-strand-displacing DNA polymerase; or
a second end repair reaction to repair fragmentation damage to the one or more ends, wherein the second end repair reaction comprises the use of a chain termination mechanism;
performing at least one of:
a tailing reaction to add an overhanging polynucleotide strand to the one or more end-repaired nucleic acid molecules, wherein the tailing reaction comprises the use of a single deoxynucleotide triphosphate (dNTP); or
a nick/gap repair reaction to fill in single-stranded nicks or gaps in the one or more end-repaired nucleic acid molecules, wherein the nick/gap repair reaction comprises the use of a DNA ligase
to generate a plurality of modified nucleic acid molecules; and
performing sequence analysis of the modified nucleic acid molecules.

34. A system comprising:
an extractor configured to extract a plurality of a plurality of nucleic acid molecules from a sample obtained from a subject;
an automated library preparation device configured to execute one or more processes that when executed perform one or more of:
a first end repair reaction to repair fragmentation damage to one or more ends of the nucleic acid molecules, wherein the first end repair reaction comprises the use of a non-strand-displacing DNA polymerase; or
a second end repair reaction to repair fragmentation damage to the one or more ends, wherein the second end repair reaction comprises the use of a chain termination mechanism;
performing at least one of:
a tailing reaction to add an overhanging polynucleotide strand to the one or more end-repaired nucleic acid molecules, wherein the tailing reaction comprises the use of a single deoxynucleotide triphosphate (dNTP); or
a nick/gap repair reaction to fill in single-stranded nicks or gaps in the one or more end-repaired nucleic acid molecules, wherein the nick/gap repair reaction comprises the use of a DNA ligase
to generate a plurality of modified nucleic acid molecules;
a sequencer configured to sequence the modified nucleic acid molecules and generate one or more sequence modified nucleic acid sequence reads representing the nucleic acid sequence of the modified nucleic acid molecule; and
a computational analysis platform including one or more processors including a memory that stores one or more processes, the processes when executed configured to analyze the modified nucleic acid reads and identify one or more alterations or epigenetic signatures in the modified nucleic acid molecules.
